(19) United States
(12) Patent Application Publication
(10) Pub. No.: US 2008/0119670 A1
(43) Pub. Date: May 22, 2008

(54) Methods for preparing glutamic acid derivatives and intermediates thereof

(75) Inventors: Timothy Doyle, Morristown, NJ (US); Joseph Zeldis, New City, NY (US); Ronald S. Michalak, Congers, NY (US); David M. Blum, Upper Saddle River, NJ (US); Mel Jennings, Highland Falls, NY (US); Julianne Blum, Upper Saddle River, NJ (US)

Correspondence Address: WilmerHale/Wyeth, 60 STATE STREET, BOSTON, MA, 02109, US

(73) Assignee: Wyeth, Madison, NJ (US)

(21) Appl. No.: 11/809,426

(22) Filed: Jun. 1, 2007

Related U.S. Application Data
(60) Provisional application No. 60/810,292, filed on Jun. 2, 2006.

(51) Int. Cl. C07C 233/01

(52) U.S. Cl. 564/211

(57) ABSTRACT

The present invention relates to novel methods for the preparation of glutamic acid derivatives and intermediates thereof, and such compounds prepared by the novel methods.

### CROSS REFERENCE TO RELATED APPLICATIONS

This application claims the benefit of priority under 35 U.S.C. §119(e) to U.S. Patent Application Ser. No. 60/810,292 filed on Jun. 2, 2006 and is hereby incorporated by reference in its entirety.

Throughout this application, various publications are referenced. The disclosures of these publications in their entireties are hereby incorporated by reference into this application in order to more fully describe the state of the art as known to those skilled therein as of the date of the invention described and claimed herein.

### FIELD OF THE INVENTION

The present invention relates to novel methods for the preparation of glutamic acid derivatives and intermediates thereof. Glutamic acid derivatives are useful as metalloproteinase inhibitors.

### BACKGROUND OF THE INVENTION

Metalloproteinases, including matrix metalloproteinases and aggrecanases, are known to have a role in the breakdown of connective tissue. Matrix metalloproteinases (“MMPs”) constitute a superfamily of proteolytic enzymes that are genetically related and capable of degrading almost all the constituents of extracellular matrix and basement membrane that restrict cell movement. Aggrecanases are members of the ADAMTS (A disintegrin and metalloproteinase with thrombospondin motifs) family of proteins. Aggrecanase-1 and aggrecanase-2 have been designated ADAMTS-4 and ADAMTS-5, respectively (Tang B. L., Int J Biochem Cell Biol 2001, 33, 33-44).

The ADAMTS family is involved in cleaving aggrecan, a cartilage component also known as the large aggregating chondroitin sulphate proteoglycan (Abbaszade I. et al., J Biol Chem 1999, 274, 23443-23450), procollagen processing (Colige A. et al., Proc Natl Acad Sci USA 1997, 94, 2374-2379), angiogenesis (Vazquez F. et al., J Biol Chem 1999, 274, 23349-23357), inflammation (Kuno K. et al., J Biol Chem 1997, 272, 556-562) and tumor invasion (Masui T. et al., J Biol Chem 1997, 272, 556-562). MMPs have been shown to cleave aggrecan as well.

The loss of aggrecan has been implicated in the degradation of articular cartilage in arthritic diseases, for example osteoarthritis is a debilitating disease which affects at least 30 million Americans. Degradation of articular cartilage and the resulting chronic pain can severely reduce quality of life. An early and important characteristic of the osteoarthritic process is loss of aggrecan from the extracellular matrix, resulting in deficiencies in the biomechanical characteristics of the cartilage. Likewise, MMPs and aggrecanases are known to play a role in many disorders in which extracellular protein degradation or destruction occurs, such as cancer, asthma, chronic obstructive pulmonary disease (“COPD”), atherosclerosis, age-related macular degeneration, myocardial infarction, corneal ulceration and other ocular surface diseases, hepatitis, aortic aneurysms, tendonitis, central nervous system diseases, abnormal wound healing, angiogenesis, restenosis, cirrhosis, multiple sclerosis, glomerulonephritis, graft versus host disease, diabetes, inflammatory bowel disease, shock, invertebral disc degeneration, stroke, osteopenia, and periodontal diseases.

The glutamic acid derivatives and the preparation thereof are disclosed in commonly assigned U.S. patent application Ser. Nos. 60/697,590, filed on Jul. 11, 2005, and 60/726,441, filed on Oct. 13, 2005, and in WO2007/008994 and WO/2007/044100.

In an attempt to synthesize a key intermediate XIVa according to Scheme 1, it was found that the Grignard step was difficult to control. For example, formation of the Grignard reagent 8 was sometimes not reliable. Additionally, it was difficult to prevent or limit the formation of homocoupling product 9. Thus, there remains a need to find a more efficient method that may be suitable for commercial manufacturing of the glutamic acid derivatives and intermediates thereof.

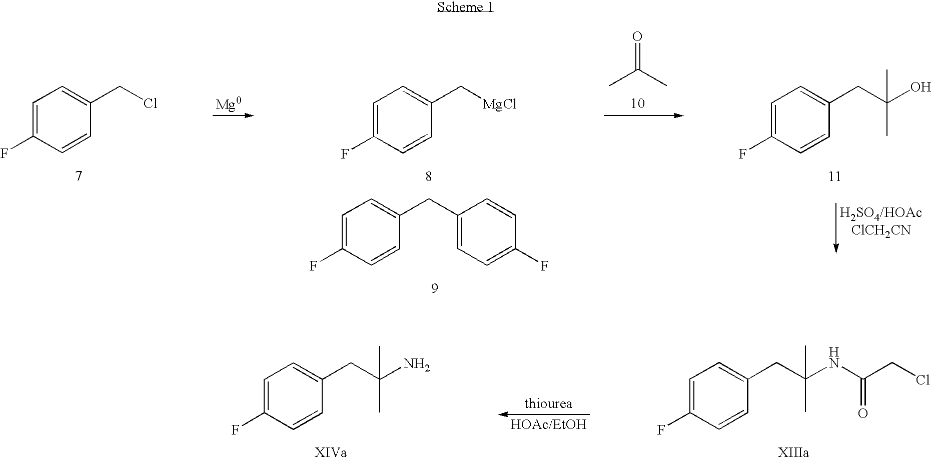

### SUMMARY OF THE INVENTION

In one aspect, the invention provides novel methods as described in the appended claims for preparing a compound of formula (I) and intermediates thereof, or pharmaceutically acceptable salt thereof,

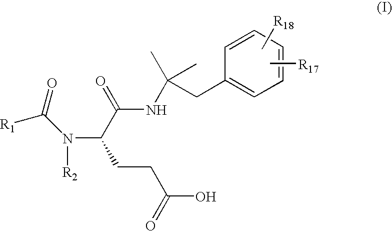

wherein:
R₁ is phenyl, heteroaryl, biphenyl, bicyclic aryl, tricyclic aryl, bicyclic heteroaryl, or tricyclic heteroaryl, each optionally substituted with one or more of R₅ or R₆, and when R₁ is substituted with more than one of R₅ or R₆, the substituents can be identical or different;
R₂ is hydrogen, (C₁-C₆) alkyl, (C₂-C₆) alkenyl, (C₂-C₆) alkynyl, —(CH₂)ₙR₁₁, —OH, or —O—(C₁-C₆) alkyl;
R₅ is aryl, heteroaryl, —(CH₂)ₙ-aryl, —(CH₂)ₙ-heteroaryl, —O-aryl, —O-heteroaryl, —S-aryl, —S-heteroaryl, —NH-aryl, —NH-heteroaryl, —C(═O)—(C₁-C₆) alkyl, —C(═O)-aryl, —C(═O)-heteroaryl, —SO₂—(C₁-C₆) alkyl, —SO₂-aryl, —SO₂-heteroaryl, —SO₂NH-aryl, —SO₂NH-heteroaryl, —NHSO₂—(C₁-C₆) alkyl, —NHSO₂-aryl, —NHSO₂-heteroaryl, —NHC(═O)-aryl, —NHC(═O)-heteroaryl, —C(═O)NH-aryl, —C(═O)NH-heteroaryl, (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, or —C(═O)NH—(C₁-C₆) cycloalkyl; each alkyl, aryl, cycloalkyl, or heteroaryl optionally substituted with one or more of R₆, and when R₅ is substituted with more than one R₆, the substituents can be identical or different;
R₆ is hydrogen, halogen, —CN, —OCF₃, —CF₃, —NO₂, —OH, —SH, —NR₇R₈, —C(═O)NR₇R₈, —NR₈C(═O)R₇, —NR₈CO₂R₇, —CO₂R₇, —C(═O)R₇, —SO₂—(C₁-C₆) alkyl, —SO₂-aryl, —SO₂-heteroaryl, —SO₂R₇, —NR₇SO₂R₈, —SO₂NR₇R₈; (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, —C(═O)NH—(C₁-C₆) cycloalkyl, heterocycloalkyl, —(C₁-C₆) alkyl-OR₇, (C₂-C₆) alkynyl, (C₂-C₆) alkenyl, —O—(C₁-C₆) alkyl-cycloalkyl, —O-alkenyl, —O—(C₁-C₆) alkyl substituted with aryl, aryl, heteroaryl, —(CH₂)ₙ-aryl, —(CH₂)ₙ-heteroaryl, —O-aryl, —O-heteroaryl, —S-aryl, or —S-heteroaryl; each alkyl, aryl, cycloalkyl, heterocycloalkyl, heteroaryl, alkenyl, or alkynyl optionally substituted with one or more of R₁₃, and when R₆ is substituted with more than one R₁₃, the substituents can be identical or different;
R₇ and R₈ are each independently hydrogen, (C₁-C₆) alkyl, aryl, heteroaryl, (C₂-C₆) alkenyl, (C₂-C₆) alkynyl, cycloalkyl, —(CH₂)ₙ-aryl, or (CH₂)ₙ-heteroaryl; or R₇ and R₈ together with the nitrogen atom to which they are attached may form a five- to seven-membered cyclic group containing up to 3 heteroatoms each independently selected from N, O, or S;
R₁₃ is halogen, —O—(C₁-C₆) alkyl, —CO₂H, —OH, —CF₃, hydrogen, (C₁-C₆) alkyl, aryl, heteroaryl, (C₂-C₆) alkenyl, (C₂-C₆) alkynyl, cycloalkyl, cycloalkyl substituted with —OH, aryl substituted with —NH₂, aryl substituted with —O—(C₁-C₆) alkyl, —(CH₂)ₙ-aryl, or —(CH₂)ₙ-heteroaryl;
R₁₇ and R₁₈ are each independently hydrogen, halogen, —CN, —OCF₃, —CF₃, —NO₂, (C₁-C₆)alkyl, (C₂-C₆)alkenyl, (C₂-C₆)alkynyl, aryl, heteroaryl, cycloalkyl, —(CH₂)ₙR₁₁, or —O—(C₁-C₆)alkyl;
   R₁₁ is aryl, heteroaryl, or cycloalkyl; and
n is 0, 1, 2, 3, or 4.

In another aspect, the invention provides glutamic acid derivatives and intermediates thereof, or pharmaceutically acceptable salt thereof prepared by such novel methods.

### FURTHER DESCRIPTION OF THE INVENTION

### Definitions

All recitations of a group, such as alkyl, are understood for the purposes of this specification to encompass both substituted and unsubstituted forms.

The term “alkyl”, as used herein, whether used alone or as part of another group, refers to a substituted or unsubstituted saturated aliphatic hydrocarbon chain and includes, but is not limited to, straight and branched chains containing from 1 to 12 carbon atoms, or in some instances, from 1 to 6 carbon atoms, unless explicitly specified otherwise. For example, methyl, ethyl, propyl, isopropyl, butyl, i-butyl and t-butyl are encompassed by the term “alkyl.” (C₁-C₆)-alkyl includes straight and branched chain aliphatic groups having from 1 to 6 carbons. Specifically included within the definition of “alkyl” are those aliphatic hydrocarbon chains that are optionally substituted. In one embodiment, an alkyl is optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The number of carbon atoms as used in the definitions herein refers to carbon backbone and carbon branching, but does not include carbon atoms of the substituents, such as alkoxy substitutions and the like.

The term “alkenyl”, as used herein, whether used alone or as part of another group, refers to a substituted or unsubstituted ethylenically unsaturated hydrocarbon chain and includes, but is not limited to, straight and branched chains having 2 to 8 carbon atoms and containing at least one double bond. In one embodiment, the alkenyl moiety has 1 or 2 double bonds. Such alkenyl moieties may exist in the E or Z conformations and the compounds of this invention include both conformations. (C₂-C₆) alkenyl includes a 2 to 6 carbon straight or branched chain having at least one carbon-carbon double bond. Specifically included within the definition of “alkenyl” are those aliphatic hydrocarbon chains that are optionally substituted. In one embodiment, a heteroatom, such as O, S or N, attached to an alkenyl is not attached to a carbon atom that is bonded to a double bond. In one embodiment, an alkenyl is optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′COR′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “alkynyl”, as used herein, whether used alone or as part of another group, refers to a hydrocarbon moiety containing at least one carbon-carbon triple bond. (C₂-C₆) alkynyl includes a 2 to 6 carbon straight or branched chain having at least one carbon-carbon triple bond. In one embodiment, an alkynyl is optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “cycloalkyl” refers to a monocyclic, bicyclic, tricyclic, fused, bridged, or spiro monovalent saturated hydrocarbon ring system, wherein the carbon atoms are located inside or outside of the ring system, e.g., of 3-15 carbon atoms. Any suitable ring position of the cycloalkyl moiety may be covalently linked to the defined chemical structure. Examples of cycloalkyl moieties include, but are not limited to, cyclopropyl, cyclopropylmethyl, cyclobutyl, cyclopentyl, cyclohexyl, cyclohexylmethyl, cyclohexylethyl, cycloheptyl, norbornyl, adamantyl, spiro[4.5]decanyl, and homologs, isomers, and the like. C₃-C₆ cycloalkyl includes monocyclic, saturated rings of 3 to 6 carbons. In one embodiment, a cycloalkyl is optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

“Heteroaryl” refers to a 5 to 6 membered aromatic heterocyclic ring which contains from 1 to 4 heteroatoms selected from the group consisting of oxygen, nitrogen, and sulfur atoms in the ring and may be fused with a carbocyclic or heterocyclic ring at any possible position (e.g. fused to one or more carbocyclic or heterocyclic rings, each having 5-8 ring atoms, the fused heterocyclic ring containing from 1 to 4 heteroatoms selected from the group consisting of oxygen, nitrogen, and sulfur atoms in the ring). Exemplary heteroaryl groups include, but are not limited to, furanyl, furazanyl, homopiperazinyl, imidazolinyl, isothiazolyl, isoxazolyl, oxadiazolyl, oxazolyl, pyrimidinyl, phenanthridinyl, pyranyl, pyrazinyl, pyrazolinyl, pyrazolyl, pyridazinyl, pyridooxazolyl, pyridoimidazolyl, pyridothiazolyl, pyridinyl, pyrimidinyl, pyrrolinyl, thiadiazinyl, thiadiazolyl, thienyl, thienothiazolyl, thienooxazolyl, thienoimidazolyl, thiophenyl, triazinyl, and triazolyl. In one embodiment, a heteroaryl is optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

“Heterocycloalkyl” refers to a saturated ring or ring system containing carbon atoms and from 1 to 4 heteroatoms selected from N, O, and S, each of the ring or ring system being 5 to 7-membered. Exemplary heterocycloalkyl groups include, but are not limited to, azepanyl, azetidinyl, aziridinyl, imidazolidinyl, morpholinyl, oxazolidinyl, piperazinyl, piperidinyl, pyrazolidinyl, pyrrolidinyl, quinuclidinyl, tetrahydrofuranyl, and thiomorpholinyl. In one embodiment, a heterocycloalkyl is optionally substituted with one or more of the following: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “aryl” as used herein as a group or part of a group refers to an aromatic carbocyclic ring system, e.g., of from 6 to 14 carbon atoms such as phenyl, which may be optionally substituted. An aryl group may be fused with a carbocyclic or heterocyclic ring at any possible position (e.g. fused to one or more carbocyclic or heterocyclic rings, each having 5-8 ring atoms, the fused heterocyclic ring containing from 1 to 4 heteroatoms selected from the group consisting of oxygen, nitrogen, and sulfur atoms in the ring). “Phenyl”, as used herein, whether used alone or as part of another group, refers to a substituted or unsubstituted phenyl group. In one embodiment, an aryl group such as phenyl is optionally substituted with one or more of the following: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “biphenyl” as used herein refers to two phenyl groups connected at one carbon site on each ring. In one embodiment, one or both phenyl groups is independently optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “biaryl” as used herein refers to two aryl groups connected at one carbon site on each ring. In one embodiment, one or both aryl groups is independently optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “bicyclic aryl” as used herein refers to two fused carbocyclic groups, wherein one or both of the carbocyclic groups is aromatic. For example, a bicyclic aryl can contain from 8 to 12 ring atoms. In one embodiment, one or both carbocyclic groups of the bicyclic aryl are independently optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “tricyclic aryl” as used herein refers to three fused carbocyclic groups, wherein two or three of the carbocyclic groups is aromatic. For example, a tricyclic aryl can contain from 13 to 18 ring atoms. In one embodiment, one or more of the carbocyclic groups of the tricyclic aryl are independently optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “bicyclic heteroaryl” as used herein refers to two fused cyclic groups, wherein one or both of the cyclic groups is aromatic and contains one to four heteroatoms selected from O, S, and N. For example, a bicyclic heteroaryl can contain from 8 to 12 ring atoms, and from 1 to 3 heteroatoms selected from O, N, and S in each ring. In one embodiment, one or both cyclic groups are independently optionally substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

The term “tricyclic heteroaryl” as used herein refers to three fused cyclic groups, wherein two or three of the cyclic groups is aromatic and at least one aromatic group contains 1 to 4 heteroatoms selected from O, S, and N. For example, a tricyclic aryl can contain from 13 to 18 ring atoms, and from 1 to 3 heteroatoms selected from O, N, and S in each ring. In one embodiment, the cyclic groups are independently substituted with one or more of the following groups: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

An optionally substituted moiety may be substituted with one or more substituents, examples of which are as illustrated herein. In one embodiment, an “optionally substituted” moiety is substituted with one or more of the following: —V-halogen, —V—(C₁-C₆)-alkyl, —V—(C₂-C₆)-alkenyl, —V—(C₂-C₆)-alkynyl, —V—N(R′)₂, methylenedioxo, ethylenedioxo, —V—NHSO₂R′, —V—NR′C(═O)R′, —V—NHCO₂R′, —V—NO₂, —V—SO₂N(R′)₂, —V—SO₂R′, —V—OR′, —V—C(═O)R′, —V—CO₂R′, —V—C(═O)N(R′)₂, or —V—CN, wherein each R′ is independently hydrogen, unsubstituted (C₁-C₆)-alkyl, or unsubstituted aryl; and wherein each V is independently a bond or (C₁-C₆)-alkyl. Each such group serving as optional substituent may contain up to 12 carbon atoms, preferably up to 6 carbon atoms.

When such moieties are substituted, for example, they may typically be mono-, di-, tri- or persubstituted. Examples for a halogen substituent include 1-bromo vinyl, 1-fluoro vinyl, 1,2-difluoro vinyl, 2,2-difluorovinyl, 1,2,2-trifluorovinyl, 1,2-dibromo ethane, 1,2 difluoro ethane, 1-fluoro-2-bromo ethane, CF₂F₃, CF₂CF₂CF₃, and the like.

The term halogen includes bromine, chlorine, fluorine, and iodine.

For the sake of simplicity, connection points (“−”) are not depicted. When an atom or compound is described to define a variable, it is understood that it is intended to replace the variable in a manner to satisfy the valency of the atom or compound. For example, if “X*” was C(R*)═C(R*), both carbon atoms form a part of the ring in order to satisfy their respective valences. Likewise, when divalent substituents are presented, it is understood that they are not limited to the order listed, for example, as used in this specification “OCH₂” encompasses CH₂O and OCH₂.

The term “amine protecting group” as used herein refers to a moiety that temporarily blocks an amine reactive site in a compound. Generally, this is done so that a chemical reaction can be carried out at another reactive site in a multifunctional compound or to otherwise stabilize the amine. In one embodiment, an amine protecting group is selectively removable by a chemical reaction. An exemplary amine protecting group is a 9-fluorenylmethoxycarbonyl protecting group. Another exemplary amine protecting group is an organoxycarbonyl group, i.e., where the amine is protected as a carbamate. Carbamates include, without limitation, t-butyl carbamate, methyl carbamate, ethyl carbamate, 2,2,2-trichloroethyl carbamate, 2-(trimethylsilyl)ethyl carbamate, 1,1-dimethyl-2,2,2-trichloroethyl carbamate, benzyl carbamate, p-methoxybenzyl carbamate, p-nitrobenzylcarbamate, p-bromobenzyl carbamate, p-chlorobenzyl carbamate, and 2,4-dichlorobenzyl carbamate. See, Greene and Wuts, Protecting Groups in Organic Synthesis, Third Edition, John Wiley & Sons (1999).

The term “carboxylic acid protecting group” as used herein refers to a moiety that temporarily blocks a carboxylic acid reactive site in a compound. Generally, this is done so that a chemical reaction can be carried out at another reactive site in a multifunctional compound or to otherwise stabilize the carboxylic acid. In one embodiment, a carboxylic acid protecting group is selectively removable by a chemical reaction. An exemplary carboxylic acid protecting group is an alkyl ester protecting group, such as a tert-butyl ester. See, Greene and Wuts, Protecting Groups in Organic Synthesis, Third Edition, John Wiley & Sons (1999).

The term “metalloproteinase-related disorder” used herein refers to a condition for which it would be beneficial to modulate activity of the metalloproteinase. Exemplary metalloproteinase-related disorders include, without limitation, arthritic disorders, osteoarthritis, cancer, rheumatoid arthritis, asthma, chronic obstructive pulmonary disease, atherosclerosis, age-related macular degeneration, myocardial infarction, corneal ulceration and other ocular surface diseases, hepatitis, aortic aneurysms, tendonitis, central nervous system diseases, abnormal wound healing, angiogenesis, restenosis, cirrhosis, multiple sclerosis, glomerulonephritis, graft versus host disease, diabetes, inflammatory bowel disease, shock, invertebral disc degeneration, stroke, osteopenia, and periodontal diseases.

The term “metalloproteinase modulator” refers to a compound that is capable of modulating the expression of a metalloproteinase. For example, a metalloproteinase modulator may enhance metalloproteinase expression. A metalloproteinase modulator may also be an inhibitor of a metalloproteinase.

The term “isolated and purified” as used herein refers to an isolate that is separate from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

As used herein, a compound of the invention includes a pharmaceutically acceptable salt thereof. The term “pharmaceutically acceptable salt” as used herein refers to a salt of an acid and a basic nitrogen atom of a compound of the present invention. Exemplary salts include, but are not limited to, sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes a hydrate of a compound of the present invention.

The term “substantially free of its corresponding opposite enantiomer” as used herein means that the compound contains no more than about 10% by weight of its corresponding opposite enantiomer. In other embodiments, the compound that is substantially free of its corresponding opposite enantiomer contains no more than about 5%, no more than about 1%, no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

The term “tautomer” as used herein refers to compounds produced by the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992).

The following abbreviations as used herein mean:

### Ac is acetate;

### ACN is acetonitrile;

### Boc is t-butyl carbamate;

### Bu is butyl;

### DMF is dimethylformamide;

### DMSO is dimethylsulfoxide;

### Et is ethyl;

### HPLC is high pressure liquid chromatography;

### IPA is isopropyl alcohol;

### Me is methyl;

### NMM is N-methylmorpholine;

### NMR is nuclear magnetic resonance;

### TBME is t-butyl methyl ether;

### TFA is trifluoroacetic acid; and

### THF is tetrahydrofuran.

### Compounds and Pharmaceutically Acceptable Salts of Compounds of the Invention

The compounds or pharmaceutically acceptable salts of compounds of the present invention can contain an asymmetric carbon atom and some of the compounds or pharmaceutically acceptable salts of compounds of the invention can contain more than one asymmetric centers or no asymmetric centers, and can thus give rise to optical isomers, diastereomers and racemic mixtures. While depicted with or without respect to a particular asymmetric center in the compounds or pharmaceutically acceptable salts of compounds of the present invention, the present invention includes such optical isomers and diastereomers, as well as racemic and resolved, enantiomerically pure R and S stereoisomers, and also other mixtures of the R and S stereoisomers and pharmaceutically acceptable salts thereof. Where a stereoisomer is provided, it can in some embodiments be provided substantially free of its corresponding opposite enantiomer.

In addition, the compounds and pharmaceutically acceptable salts of compounds of the present invention can exist as tautomers. Such tautomers can be transient or isolatable as a stable product. These tautomers are within the scope of the present invention.

Prodrugs of the compounds or pharmaceutically acceptable salts of compounds are also within the scope of the present invention.

### Further Illustration of the Present Invention

For compounds of formulas (I) through (XIV) and all reagents used in the preparation thereof, and throughout the specification, the symbols are defined as follows unless otherwise noted:
R₁ is phenyl, heteroaryl, biphenyl, bicyclic aryl, tricyclic aryl, bicyclic heteroaryl, or tricyclic heteroaryl, each optionally substituted with one or more of R₅ or R₆, and when R₁ is substituted with more than one of R₅ or R₆, the substituents can be identical or different;
R₂ is hydrogen, (C₁-C₆) alkyl, (C₂-C₆) alkenyl, (C₂-C₆) alkynyl, —(CH₂)ₙR₁₁, —OH, or —O—(C₁-C₆) alkyl;
R₅ is aryl, heteroaryl, —(CH₂)ₙ-aryl, —(CH₂)ₙ-heteroaryl, —O-aryl, —O-heteroaryl, —S-aryl, —S-heteroaryl, —NH-aryl, —NH-heteroaryl, —C(═O)—(C₁-C₆) alkyl, —C(═O)-aryl, —C(═O)-heteroaryl, —SO₂—(C₁-C₆) alkyl, —SO₂-aryl, —SO₂-heteroaryl, —SO₂NH-aryl, —SO₂NH-heteroaryl, —NHSO₂—(C₁-C₆) alkyl, —NHSO₂-aryl, —NHSO₂-heteroaryl, —NHC(═O)-aryl, —NHC(═O)-heteroaryl, —C(═O)NH-aryl, —C(═O)NH-heteroaryl, (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, or —C(═O)NH—(C₁-C₆) cycloalkyl; each alkyl, aryl, cycloalkyl, or heteroaryl optionally substituted with one or more of R₆, and when R₅ is substituted with more than one R₆, the substituents can be identical or different;
R₆ is hydrogen, halogen, —CN, —OCF₃, —CF₃, —NO₂, —OH, —SH, —NR₇R₈, —C(═O)NR₇R₈, —N₈C(═O)R₇, —NR₈CO₂R₇, —CO₂R₇, —C(═O)R₇, —SO₂—(C₁-C₆) alkyl, —SO₂-aryl, —SO₂-heteroaryl, —SO₂R₇, —NR₇SO₂R₈, SO₂NR₇R₈; (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, —C(═O)NH—(C₁-C₆) cycloalkyl, heterocycloalkyl, —(C₁-C₆) alkyl-OR₇, (C₂-C₆) alkynyl, (C₂-C₆) alkenyl, —O—(C₁-C₆) alkyl-cycloalkyl, —O-alkenyl, —O—(C₁-C₆) alkyl substituted with aryl, aryl, heteroaryl, —(CH₂)ₙ-aryl, —(CH₂)ₙ-heteroaryl, —O-aryl, —O-heteroaryl, —S-aryl, or —S-heteroaryl; each alkyl, aryl, cycloalkyl, heterocycloalkyl, heteroaryl, alkenyl, or alkynyl optionally substituted with one or more of R₁₃, and when R₆ is substituted with more than one R₁₃, the substituents can be identical or different;
R₇ and R₈ are each independently hydrogen, (C₁-C₆) alkyl, aryl, heteroaryl, (C₂-C₆) alkenyl, (C₂-C₆) alkynyl, cycloalkyl, —(CH₂)ₙ-aryl, or —(CH₂)ₙ-heteroaryl; or R₇ and R₈ together with the nitrogen atom to which they are attached may form a five- to seven-membered cyclic group containing up to 3 heteroatoms each independently selected from N, O, or S;
R₁₁ is aryl, heteroaryl, or cycloalkyl;
R₁₃ is halogen, —O—(C₁-C₆) alkyl, —CO₂H, —OH, —CF₃, hydrogen, (C₁-C₆) alkyl, aryl, heteroaryl, (C₂-C₆) alkenyl, (C₂-C₆) alkynyl, cycloalkyl, cycloalkyl substituted with —OH, aryl substituted with —NH₂, aryl substituted with —O—(C₁-C₆) alkyl, —(CH₂)_{n-aryl, or —(CH}₂)ₙ-heteroaryl;
R₁₆ is (C₁-C₆) alkyl;
R₁₇ and R₁₈ are each independently hydrogen, halogen, —CN, —OCF₃, —CF₃, —NO₂, (C₁-C₆)alkyl, (C₂-C₆)alkenyl, (C₂-C₆)alkynyl, aryl, heteroaryl, cycloalkyl, —(CH₂)ₙR₁₁, or —O—(C₁-C₆)alkyl;
PG₁ is an amine protecting group;
PG₂ is a carboxylic acid protecting group; and
n is 0, 1, 2, 3, or 4.

The compounds of formula (I) through (XIV) include enantiomerically pure compounds and/or sensitive protecting groups which may be labile to certain reaction conditions. Advantageously, the present invention provides methods for preparing such compounds substantially free of their corresponding opposite enantiomers and without disturbing the protecting groups when such groups are needed.

In one embodiment, the present invention is directed to a method of preparing a compound of formula (I),

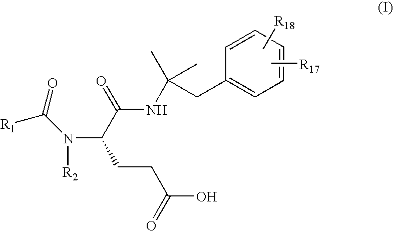

comprising:
(a) treating a compound of formula (VIII),

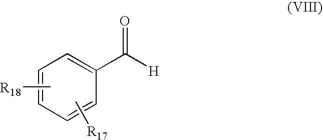

with an organometallic compound selected from the group consisting of isopropyl magnesium halide, isopropyl lithium, diisopropyl zinc and isopropyl zinc halide, preferably isopropyl magnesium chloride,
to provide a compound of formula (X);

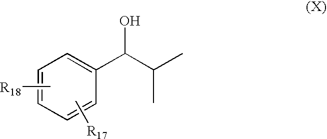

(b) treating the compound of formula (X) with at least one acid and chloroacetonitrile under conditions to effect a Ritter reaction (both sequential administration with no particular order and co-administration of said acid and chloroacetonitrile are contemplated),
to give a compound of formula (XIII),

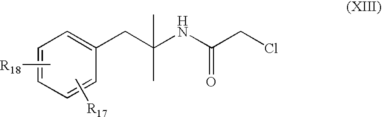

(c) treating the compound of formula (XIII) with a base and/or thiourea, preferably thiourea, to give a compound of formula (XIV);

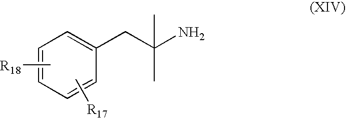

(d) optionally treating the compound of formula (XIV) with a pharmaceutically acceptable acid to provide a corresponding pharmaceutically acceptable salt of the compound of formula (XIV);
(e) converting the compound having formula (XIV) or its pharmaceutically acceptable salt into the compound of formula (I) or a pharmaceutically acceptable salt thereof; and optionally step (e) further comprises the following steps:
(f) treating the compound of formula (XIV) or its pharmaceutically acceptable salt with a compound selected from the group consisting of:
   (i) a compound of formula (IV),

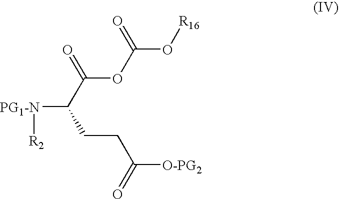

(ii) a compound of formula (IVb), and

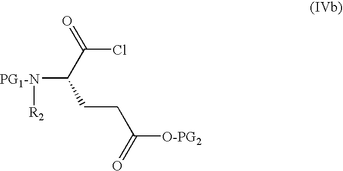

(iii) a compound of formula (II),

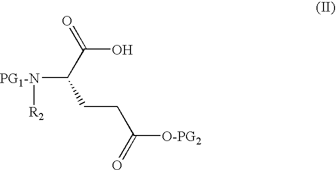

to give a compound of formula (V);

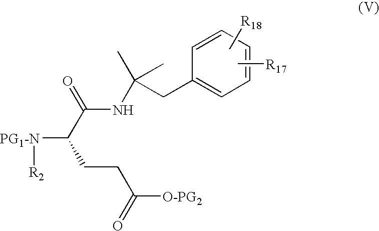

(g) removing the amine protecting group of the compound of formula (V) to give a compound of formula (VI);

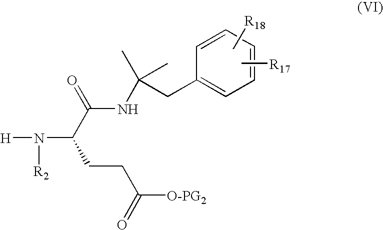

(h) treating the compound of formula (VI) with an acid chloride having the formula R₁C(═O)Cl in the presence of a base to give a compound of formula (VII); and

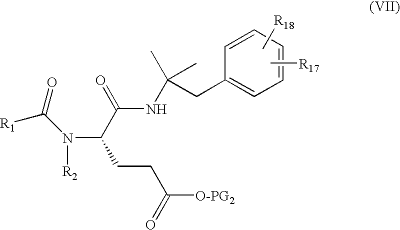

(i) removing the carboxylic acid protecting group of the compound of formula (VII) to give a compound of formula (I).

In another embodiment, the present invention is directed to a method for preparing a compound of the formula (XIII),

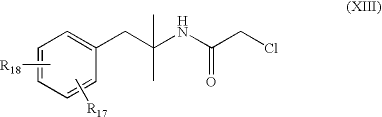

comprising:
(a) treating a compound of formula (VIII),

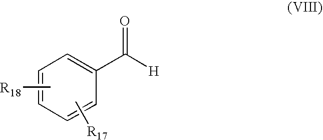

with an organometallic compound selected from the group consisting of isopropyl magnesium halide, isopropyl lithium, diisopropyl zinc and isopropyl zinc halide, preferably isopropyl magnesium chloride,
to provide a compound of formula (X); and

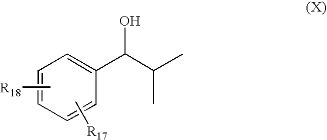

(b) treating the compound of formula (X) with at least one acid and chloroacetonitrile under conditions to effect a Ritter reaction (both sequential administration with no particular order and co-administration of said acid and chloroacetonitrile are contemplated).

In yet another embodiment, the present invention is directed to a method for preparing a compound of the formula (XIV) or a pharmaceutically acceptable salt thereof,

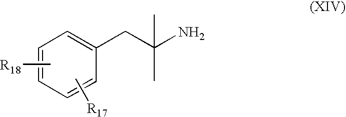

comprising:
(a) treating a compound of formula (VIII),

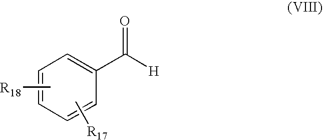

with an organometallic compound selected from the group consisting of isopropyl magnesium halide, isopropyl lithium, diisopropyl zinc and isopropyl zinc halide, preferably isopropyl magnesium chloride,
to provide a compound of formula (X); and

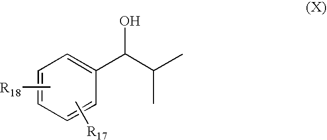

(b) treating the compound of formula (X) with at least one acid and chloroacetonitrile under conditions to effect a Ritter reaction (both sequential administration with no particular order and co-administration of said acid and chloroacetonitrile are contemplated),
to give a compound of formula (XIII);

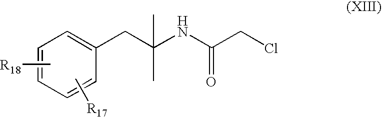

(c) treating the compound of formula (XIII) with a base and/or thiourea, preferably thiourea, to give the compound of formula (XIV); and
(d) optionally treating the compound of formula (XIV) with a pharmaceutically acceptable acid to provide the corresponding pharmaceutically acceptable salt of the compound of formula (XIV).

In a further embodiment, the present invention is directed to a method for preparing a compound of formula (XIIIa),

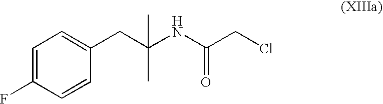

comprising:
(a) treating a compound of formula (VIIIa),

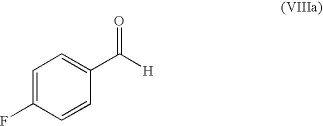

with an organometallic compound selected from the group consisting of isopropyl magnesium halide, isopropyl lithium, diisopropyl zinc and isopropyl zinc halide, preferably isopropyl magnesium chloride,
to provide a compound of formula (Xa); and

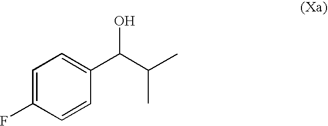

(b) treating the compound of formula (Xa) with at least one acid and chloroacetonitrile under conditions to effect a Ritter reaction (both sequential administration with no particular order and co-administration of said acid and chloroacetonitrile are contemplated).

In another embodiment, the present invention is directed to a method for preparing a compound of formula (XIVa), or a pharmaceutically acceptable salt thereof,

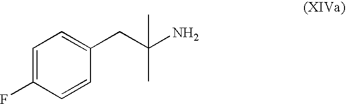

comprising:
(a) treating a compound of formula (VIIIa),

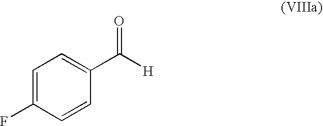

with an organometallic compound selected from the group consisting of isopropyl magnesium halide, isopropyl lithium, diisopropyl zinc and isopropyl zinc halide, preferably isopropyl magnesium chloride,
to provide a compound of formula (Xa);

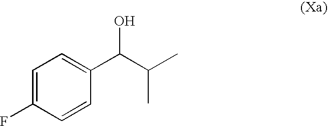

(b) treating the compound of formula (Xa) with at least one acid and chloroacetonitrile under conditions to effect a Ritter reaction (both sequential administration with no particular order and co-administration of said acid and chloroacetonitrile are contemplated),
to give a compound of formula (XIIIa);

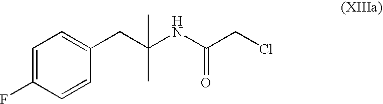

(c) treating the compound of formula (XIIIa) with a base and/or thiourea, preferably thiourea, to give a compound of formula (XIVa); and

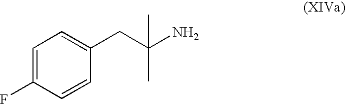

(d) optionally treating the compound of formula (XIVa) with a pharmaceutically acceptable acid to provide a corresponding pharmaceutically acceptable salt of the compound of formula (XIVa).

In yet another embodiment, the present invention is directed to a method for preparing a compound of formula (Ia),

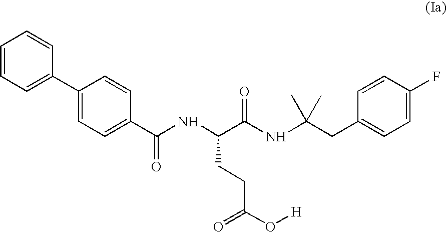

comprising:
(a) treating a compound of formula (VIIIa),

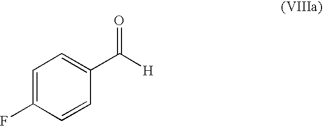

with an organometallic compound selected from the group consisting of isopropyl magnesium halide, isopropyl lithium, diisopropyl zinc and isopropyl zinc halide, preferably isopropyl magnesium chloride,
to provide a compound of formula (Xa);

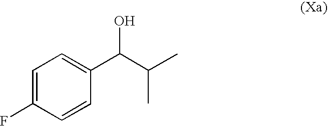

(b) treating the compound of formula (Xa) with at least one acid and chloroacetonitrile under conditions to effect a Ritter reaction (both sequential administration with no particular order and co-administration of said acid and chloroacetonitrile are contemplated),
to give a compound of formula (XIIIa);

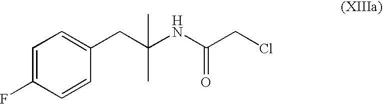

(c) treating the compound of formula (XIIIa) with a base and/or thiourea, preferably thiourea, to give a compound of formula (XIVa); and

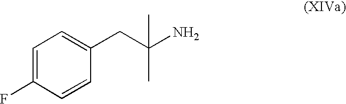

(d) optionally treating the compound of formula (XIVa) with a pharmaceutically acceptable acid to provide a corresponding pharmaceutically acceptable salt of the compound of formula (XIVa);
(e) converting the compound having formula (XIVa) or its pharmaceutically acceptable salt into the compound of formula (Ia) or a pharmaceutically acceptable salt thereof, and optionally step (e) further comprises the following steps:
(f) treating the compound of formula (XIVa) or its pharmaceutically acceptable salt with a compound selected from the group consisting of:
   (i) a compound of formula (IVa),

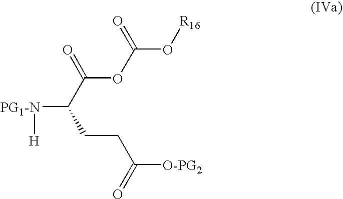

(ii) a compound of formula (IVc), and

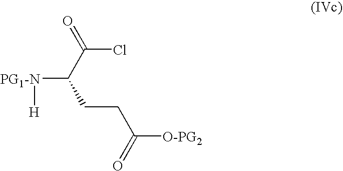

(iii) a compound of formula (IIa),

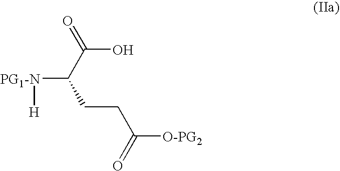

to give a compound of formula (Va);

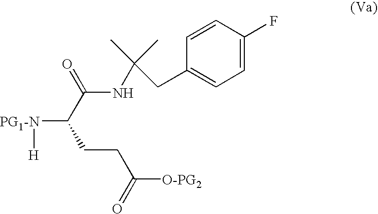

(g) removing the amine protecting group of the compound of formula (Va) to give a compound of formula (VIa);

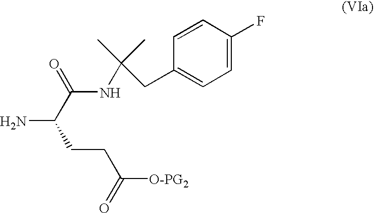

(h) treating the compound of formula (VIa) with an acid chloride having the formula of

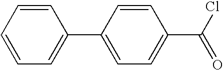

in the presence of a base to give a compound of formula (VIIa); and

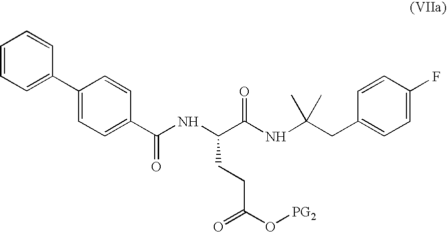

(i) removing the carboxylic acid protecting group of the compound of formula (VIIa) to give a compound of formula (Ia).

In a further embodiment, the present invention is directed to a compound of formula (XIIIa)

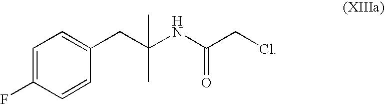

In yet another embodiment, the present invention is directed to a compound of formula (XIII),

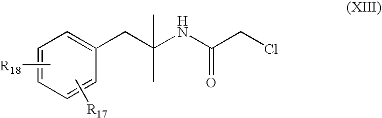

prepared by the method comprising:
(a) treating a compound of formula (VIII),

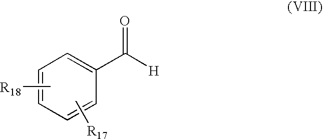

with an organometallic compound selected from the group consisting of isopropyl magnesium halide, isopropyl lithium, diisopropyl zinc and isopropyl zinc halide, preferably isopropyl magnesium chloride,
to provide a compound of formula (X); and

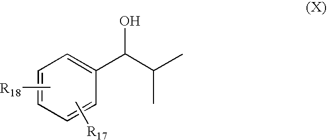

(b) treating the compound of formula (X) with at least one acid and chloroacetonitrile under conditions to effect a Ritter reaction (both sequential administration with no particular order and co-administration of said acid and chloroacetonitrile are contemplated).

In yet another embodiment, the present invention is directed to a compound of formula (XIV), or a pharmaceutically acceptable salt thereof,

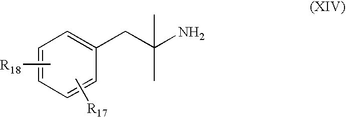

prepared by the method comprising:
(a) treating a compound of formula (VIII),

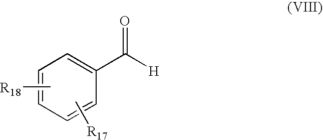

with an organometallic compound selected from the group consisting of isopropyl magnesium halide, isopropyl lithium, diisopropyl zinc and isopropyl zinc halide, preferably isopropyl magnesium chloride,
to provide a compound of formula (X); and

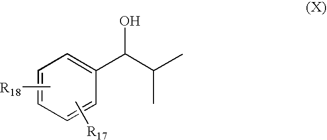

(b) treating the compound of formula (X) with at least one acid and chloroacetonitrile under conditions to effect a Ritter reaction (both sequential administration with no particular order and co-administration of said acid and chloroacetonitrile are contemplated), to give a compound of formula (XIII);

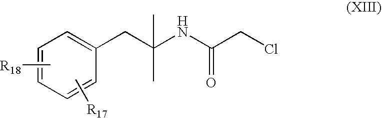

(c) treating the compound of formula (XIII) with a base and/or thiourea, preferably thiourea, to give the compound of formula (XIV); and
(b) optionally treating the compound of formula (XIV) with a pharmaceutically acceptable acid to provide the corresponding pharmaceutically acceptable salt of the compound of formula (XIV).

In yet another embodiment, the present invention is directed to a compound of formula (I),

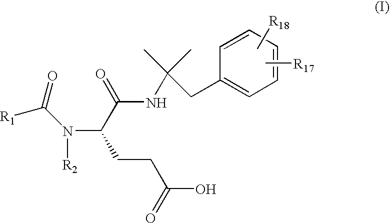

prepared by the method comprising:
(a) treating a compound of formula (VIII),

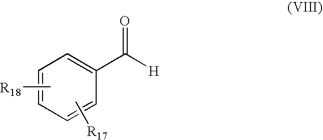

with an organometallic compound selected from the group consisting of isopropyl magnesium halide, isopropyl lithium, diisopropyl zinc and isopropyl zinc halide, preferably isopropyl magnesium chloride,
to provide a compound of formula (X);

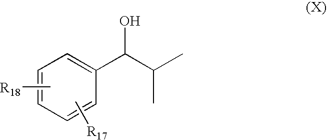

(b) treating the compound of formula (X) with at least one acid and chloroacetonitrile under conditions to effect a Ritter reaction (both sequential administration with no particular order and co-administration of said acid and chloroacetonitrile are contemplated), to give a compound of formula (XIII),

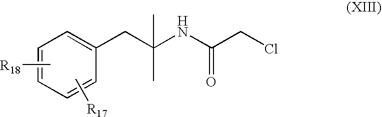

(c) treating the compound of formula (XIII) with a base and/or thiourea, preferably thiourea, to give a compound of formula (XIV);

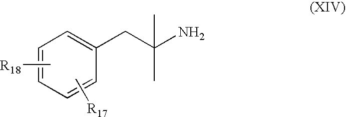

(d) optionally treating the compound of formula (XIV) with a pharmaceutically acceptable acid to provide a corresponding pharmaceutically acceptable salt of the compound of formula (XIV);
(e) converting the compound having formula (XIV) or its pharmaceutically acceptable salt into the compound of formula (I) or a pharmaceutically acceptable salt thereof; and optionally step (e) further comprises the following steps:
(f) treating the compound of formula (XIV) or its pharmaceutically acceptable salt with a compound selected from the group consisting of:
   (i) a compound of formula (IV),

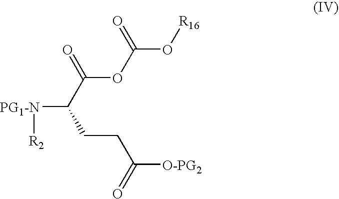

(ii) a compound of formula (IVb), and

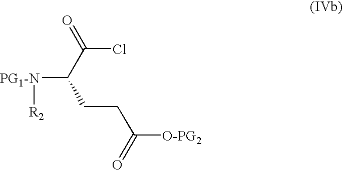

(iii) a compound of formula (II),

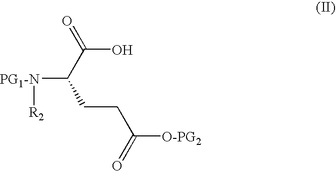

to give a compound of formula (V);

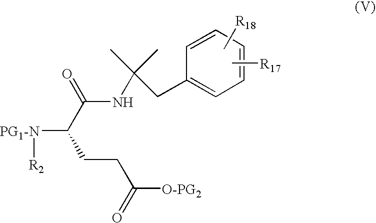

(g) removing the amine protecting group of the compound of formula (V) to give a compound of formula (VI);

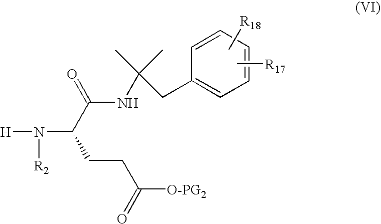

(h) treating the compound of formula (VI) with an acid chloride having the formula R₁C(═O)Cl in the presence of a base to give a compound of formula (VII); and

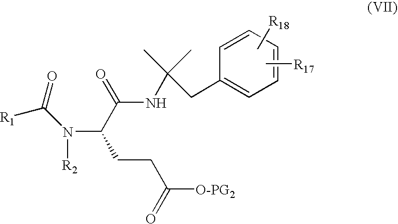

(i) removing the carboxylic acid protecting group of the compound of formula (VII) to give a compound of formula (I).

In yet another embodiment, the present invention is directed to a compound of formula (XIIIa),

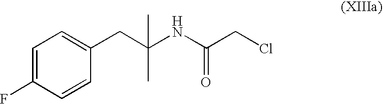

prepared by the method comprising:
(a) treating a compound of formula (VIIIa),

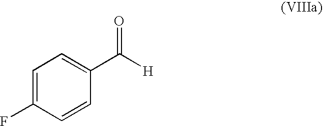

with an organometallic compound selected from the group consisting of isopropyl magnesium halide, isopropyl lithium, diisopropyl zinc and isopropyl zinc halide, preferably isopropyl magnesium chloride,
to provide a compound of formula (Xa); and

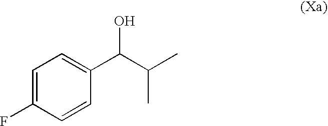

(b) treating the compound of formula (Xa) with at least one acid and chloroacetonitrile under conditions to effect a Ritter reaction (both sequential administration with no particular order and co-administration of said acid and chloroacetonitrile are contemplated).

In yet another embodiment, the present invention is directed to a compound of formula (XIVa), or a pharmaceutically acceptable salt thereof,

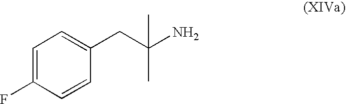

prepared by the method comprising:
(a) treating a compound of formula (VIIIa),

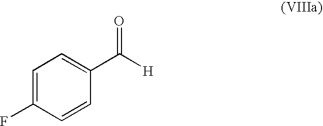

with an organometallic compound selected from the group consisting of isopropyl magnesium halide, isopropyl lithium, diisopropyl zinc and isopropyl zinc halide, preferably isopropyl magnesium chloride,
to provide a compound of formula (Xa);

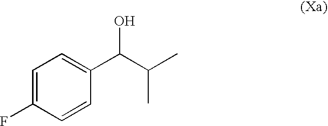

(b) treating the compound of formula (Xa) with at least one acid and chloroacetonitrile under conditions to effect a Ritter reaction (both sequential administration with no particular order and co-administration of said acid and chloroacetonitrile are contemplated), to give a compound of formula (XIIIa);

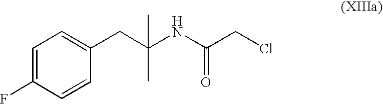

(c) treating the compound of formula (XIIIa) with a base and/or thiourea, preferably thiourea, to give a compound of formula (XIVa); and

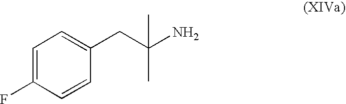

(d) optionally treating the compound of formula (XIVa) with a pharmaceutically acceptable acid to provide a corresponding pharmaceutically acceptable salt of the compound of formula (XIVa).

In yet another embodiment, the present invention is directed to a compound of formula (Ia),

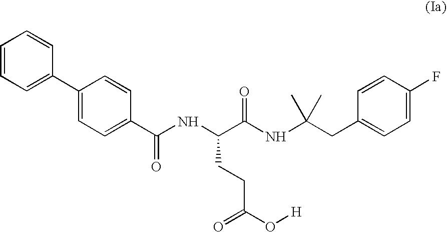

prepared by the method comprising:
(a) treating a compound of formula (VIIIa),

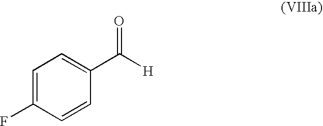

with an organometallic compound selected from the group consisting of isopropyl magnesium halide, isopropyl lithium, diisopropyl zinc and isopropyl zinc halide, preferably isopropyl magnesium chloride,
to provide a compound of formula (Xa);

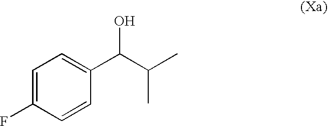

(b) treating the compound of formula (Xa) with at least one acid and chloroacetonitrile under conditions to effect a Ritter reaction (both sequential administration with no particular order and co-administration of said acid and chloroacetonitrile are contemplated), to give a compound of formula (XIIIa);

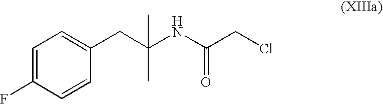

(c) treating the compound of formula (XIIIa) with a base and/or thiourea, preferably thiourea, to give a compound of formula (XIVa); and

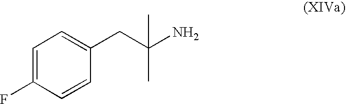

(d) optionally treating the compound of formula (XIVa) with a pharmaceutically acceptable acid to provide a corresponding pharmaceutically acceptable salt of the compound of formula (XIVa);
(e) converting the compound having formula (XIVa) or its pharmaceutically acceptable salt into the compound of formula (Ia) or a pharmaceutically acceptable salt thereof, and optionally step (e) further comprises the following steps:
(f) treating the compound of formula (XIVa) or its pharmaceutically acceptable salt with a compound selected from the group consisting of:
   (i) a compound of formula (IVa),

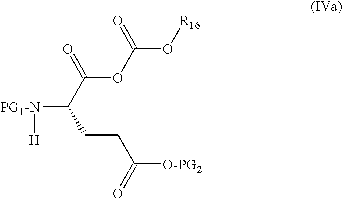

(ii) a compound of formula (IVc), and

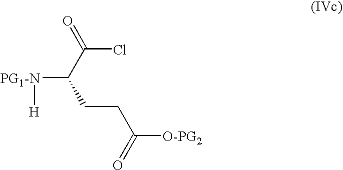

(iii) a compound of formula (IIa),

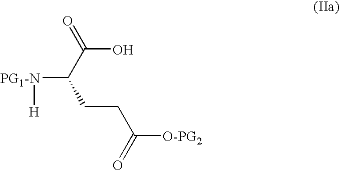

to give a compound of formula (Va);

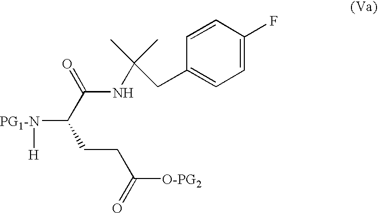

(g) removing the amine protecting group of the compound of formula (Va) to give a compound of formula (VIa);

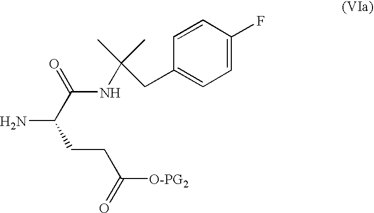

(h) treating the compound of formula (VIa) with an acid chloride having the formula of

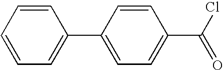

in the presence of a base to give a compound of formula (VIIa); and

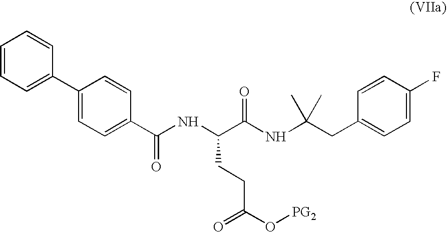

(i) removing the carboxylic acid protecting group of the compound of formula (VIIa) to give a compound of formula (Ia).

### Methods of Preparation

The compounds and pharmaceutically acceptable salts of compounds of the present invention can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

Scheme 2 demonstrates the synthesis of the compound of formula (XIV) from the compound of formula (VIII). The compound of formula (VIII) is commercially available or can be prepared by a person of ordinary skill in the art. The compound of formula (VIII) can react with an organometallic compound such as isopropyl magnesium halide, isopropyl lithium, diisopropyl zinc and isopropyl zinc halide, preferably isopropyl magnesium chloride, to give a compound of formula (X). Preferred halides for said organometallic compound are bromide and chloride. Treatment of the compound of formula (X) with at least one acid (such as sulfuric acid, or glacial acetic acid and sulfuric acid) and chloroacetonitrile under conditions to effect a Ritter reaction to provide a compound of formula (XIII). Said acid and chloroacetonitrile can be administered sequentially with no particular order, or can be co-administered. Presumably, the acid treatment of the benzylic alcohol of formula (X) affords a carbocation of formula (XI), which rearranges to a carbocation of formula (XII). Optionally, the benzylic alcohol of formula (X) can be converted to a corresponding styrene derivative (i.e., the elimination product of the hydroxyl group) before forming the carbocation of formula (XII) upon treating with at least one acid.

The carbocation of formula (XII) can further react with chloroacetonitrile to give a chloroacetamide of formula (XIII), which can be converted to a tertiary amine of formula (XIV) by reacting with a base and/or thiourea, preferably just thiourea. Said base preferably is an aqueous base, such as sodium or potassium hydroxide. Optionally, the tertiary amine of formula (XIV) can be further converted a pharmaceutically acceptable salt by reacting with a pharmaceutically acceptable acid, such as hydrochloric acid.

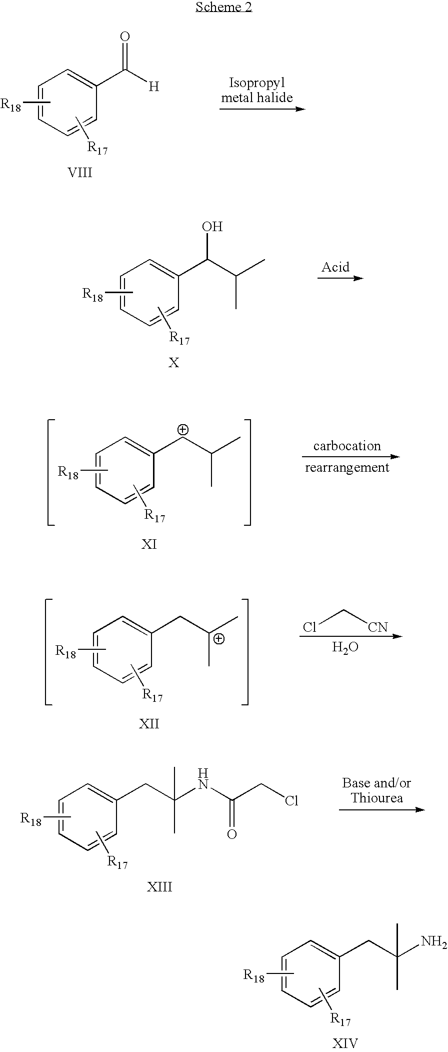

Scheme 3 describes a synthesis of a compound of formula (I) from the compound of formula (XIV), or a pharmaceutically acceptable salt thereof. The amine of formula (XIV) can be coupled with (i) a compound of formula (IV); or (ii) a compound of formula (IVb), or (iii) a compound of formula (II), in the presence of a base and/or a peptide coupling reagent, to afford a compound of formula (V). Non-limiting examples of the peptide coupling reagent include N,N′-Dicyclohexylcarbodiimide [DCC], 1-(3-Dimethylaminopropyl)-3-ethylcarbodiimide hydrochloride [EDCI], and those recited in Bodansky and Bodansky, Practice of Peptide Synthesis, 2nd ed., Springer-Verlag, Berlin (1994), the disclosure of which is incorporated herein by reference in its entirety. Treatment of the compound of formula (V) with an amine base can cleave the amine protecting group of PG₁ to provide a compound of formula (VI). A variety of amine bases may be used, including for example, diethylamine, piperidine, morpholine, dicyclohexylamine, p-dimethylaminopyridine, or diisopropylethylamine in a solvent, such as acetonitrile or DMF.

Coupling of the compound of formula (VI) with an acid of formula R₁COCl in the presence of a base affords a compound of formula (VII). The carboxylic acid protecting group of the compound of formula (VII) can be cleaved to give the compound of formula (I). The cleavage step can be carried out using TFA, NaOH, LiOH, potassium carbonate, or the like.

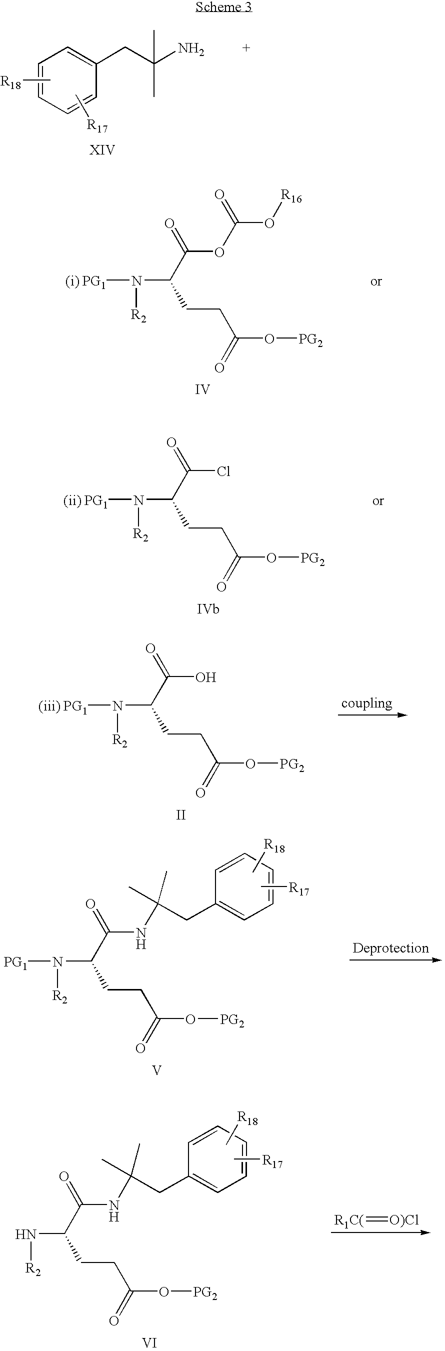

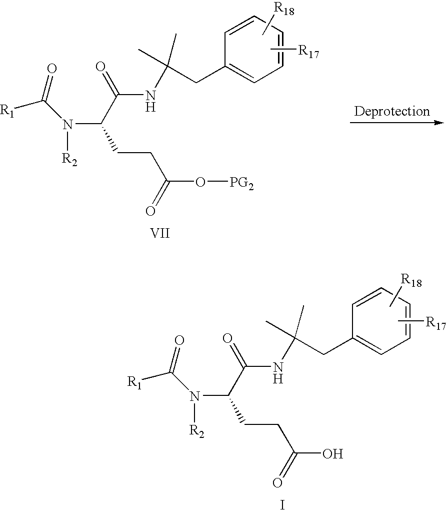

Scheme 4 further demonstrates the synthesis of a compound of formula (XIVa) or pharmaceutically acceptable salt thereof from a compound of formula (VIIIa), using a method analogous to that described in Scheme 2.

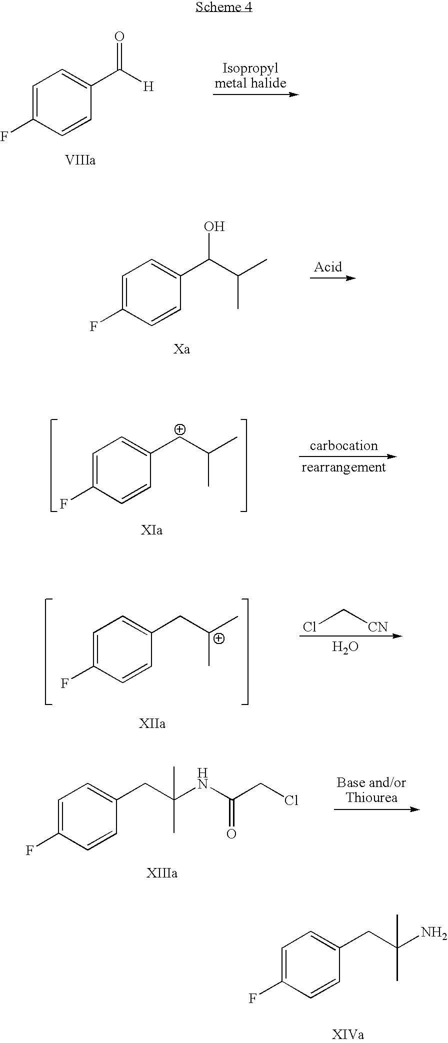

Scheme 5 further describes the synthesis of a compound of formula (Ia) from a compound of formula (XIVa) or pharmaceutically acceptable salt thereof, using a method analogous to that described in Scheme 3. The carboxylic acid protecting group of the compound of formula (VIIa) can be cleaved to give the compound of formula (Ia). The cleavage step can be achieved by using TFA. Alternatively, the cleavage step can be carried out via hydrolysis by using a base such as NaOH, LiOH, potassium carbonate, or the like. Applicants do not wish to be bound by any mechanism through which the deprotection step is achieved.

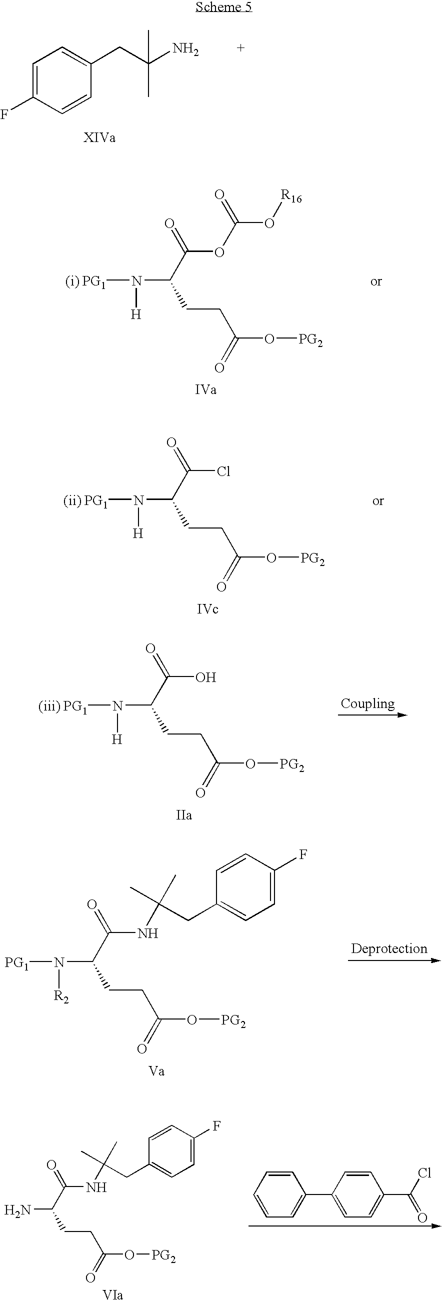

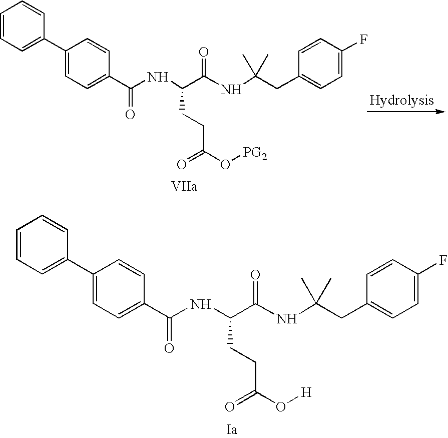

One of ordinary skill in the art will recognize that Schemes 2 through 5 can be adapted to produce other compounds and pharmaceutically acceptable salts of compounds according to the present invention.

### EXAMPLES

The following HPLC conditions were used for examples shown below unless otherwise noticed:

| Sample | Final product: Dissolve 2-3 mg of solid in 2 mL | | |
|---|---|---|---|
| Preparation: | of acetonitrile. | | |
| In-process | Dissolve 1-2 drops of reaction mixture in 2 mL | | |
| samples: | of 50:50 acetonitrile: water containing | | |
| | 1-2 drops of acetic acid. | | |
| Column: | Agilent Eclipse XDB-C8, 5μ, 4.6 × 150 mm | | |
| | Temperature = 25° C. | | |
| Flow: | 1.5 mL/min | | |
| Mobile Phase: | Solvent A = 95% acetonitrile/5% | | |
| | H₂O/0.05% TFA | | |
| | Solvent B = 95% H₂O/5% acetonitrile/0.05% TFA | | |
| Timetable: | Time | Solvent A | Solvent B |
| | 0.00 | 10.0% | 90.0% |
| | 15.00 min | 100.0% | 0.00% |

| | |
|---|---|
| Stoptime = | 20.0 min |
| Posttime = | 5.0 min |
| Detector: | Signal = 220 nm, Bw = 4; |
| | Reference = 360 nm, Bw = 100 |
| | Peakwidth >0.1 min |
| | Slit = 4 nm |
| Injection = | 5 μl |

### Example 1

### Preparation of 1-(4-fluorophenyl)-2-methyl-1-propanol

4-Fluorobenzaldehyde (186.0 grams, 1.50 moles) was added dropwise to a solution of isopropylmagnesium chloride in tetrahydrofuran (2.0 M, 787.8 grams, 1.62 moles) maintained at about 0°-10° C. After completion of the addition, the reaction mixture was allowed to stir at 0°-10° C. for about 2 hr.

The reaction mixture was transferred over about 70 minutes into a 3-liter, 4-neck roundbottom flask (equipped with a mechanical agitator, temperature probe, and nitrogen inlet) containing a solution of glacial acetic acid (126 ml) in water (1.06 L) maintained at about 5°-15° C. The flask and transfer lines were rinsed into the quench vessel with THF. The resulting two-phase mixture was allowed to stir for about 15 minutes at about 5°-15° C., and then the phases were separated. The organic phase was then washed with 5% NaCl solution.

The organic phase was concentrated under reduced pressure. Glacial acetic acid (253 grams, 242 ml) was added to the concentrate, which was then further concentrated under reduced pressure to give 1-(4-fluorophenyl)-2-methyl-1-propanol as a solution in acetic acid (359 grams, 338 ml). This solution was used in the next synthetic step without further purification. HPLC retention time of 1-(4-fluorophenyl)-2-methyl-1-propanol=9.65 min.

### Example 2

### Preparation of Chloro-N-[2-(4-fluorophenyl)-1,1-dimethylethyl]acetamide

A 500-ml, 4-neck roundbottom flask was equipped with a mechanical agitator, temperature probe, nitrogen inlet, and 125-ml liquid addition funnel. To the flask was charged a solution of 1-(4-fluorophenyl)-2-methyl-1-propanol in acetic acid (135 grams, 127 mL) as prepared in the first step. Glacial acetic acid (90 grams, 85.8 ml) was then charged to the flask. The resulting mixture was cooled to about 0°-5° C. The 125-ml liquid addition funnel was charged with 32% sulfuric acid (83.7 grams). The sulfuric acid was added dropwise to the reaction mixture. During this addition, the reaction temperature was maintained at about 0°-10° C. After completion of the addition, the reaction mixture was allowed to warm to about 20°-25° C. over about 40 minutes, and then stirred at about 20°-25° C. for over 20 hr. The reaction mixture was then transferred over about 55 minutes into a 500-ml round bottom flask containing chloroacetonitrile (63.0 grams). During this addition, the reaction temperature was maintained between about 20°-30° C. After completion of the addition, the resulting mixture was allowed to stir at about 20°-30° C. for about 3 hr. Completion of the reaction was assessed by HPLC.

When the reaction was complete, the mixture was transferred over 20 minutes into a 3-liter round bottom flask containing a mixture of water (470 ml), toluene (62 ml), and heptane (62 ml) at about 0°-5° C. During the transfer, the drown-out mixture was maintained between about 0°-10° C. After the transfer was completed, the resulting two-phase mixture was agitated for about five minutes, and then allowed to phase separate. The lower aqueous phase was removed, and then the organic phase was washed successively with 5% sodium chloride solution, followed by water. Heptane (178 grams) was added to the organic phase, and then the mixture was distilled under reduced pressure to remove approximately 53 ml of distillate. The batch was allowed to slowly cool to about 20°-25° C. When the mixture reached about 29° C., the product began to crystallize. The mixture was allowed to stir at about 20°-25° C. for about 16 hours, and then cooled to about 0°-5° C. The mixture was filtered and the product was washed with heptane. The product was dried to give chloro-N-[2-(4-fluorophenyl)-1,1-dimethylethyl]acetamide as a white solid (60.11 grams, 96.4% HPLC area). HPLC retention time=10.32 min. ¹H NMR (CDCl₃, 300 MHz): δ 7.26 (s, 1H), 7.13-7.06 (m, 2H), 7.01-6.95 (m, 2H), 6.17 (br s, 1H), 3.95 (s, 2H), 3.03 (s, 2H), 1.36 (s, 6H).

### Example 3

### Preparation of 2-(4-fluorophenyl)-1′-dimethylethylamine hydrochloride

A solution of chloro-N-[2-(4-fluorophenyl)-1,1-dimethylethyl]acetamide (71.4 g, 0.293 mol) in ethanol (125 ml) and acetic acid (55 ml) was added dropwise to a stirred suspension of thiourea (26.7 g, 0.351 mol) in ethanol (125 ml) at about 78°-86° C. The resulting mixture was allowed to stir at about 80°-86° C. for about 4 hours. The reaction was monitored for completion by HPLC.

When the reaction was complete, the mixture was cooled to about 20°-25° C. and then filtered. The reaction flask and filter cake were rinsed with ethanol, and then the filtrate was concentrated under vacuum. Water (about 200 ml) and toluene (about 200 ml) were added and the phases were thoroughly mixed. The phases were separated, and the upper organic phase was discarded. Toluene (about 400 ml) was added to the lower aqueous phase, and the resulting mixture was cooled to about 5°-10° C. Sodium hydroxide solution (50% w/w, about 50 ml) was added dropwise to adjust the pH of the aqueous phase to about 13-14. The phases were separated, and then the upper organic layer was washed with water. Hydrochloric acid solution (37%, 53 g, 44 ml, 0.44 mol) was added to the organic solution, and then the mixture was concentrated under vacuum to approximately 500 ml volume. During the concentration, water was removed, and the product began to precipitate. The product mixture was cooled to about 15° C. and then filtered. The product cake was rinsed with toluene and then dried to give 2-(4-fluorophenyl)-1,1-dimethylethylamine hydrochloride (43.6 grams, 87% HPLC area). HPLC Retention time=4.6 min. ¹H NMR: (DMSO-d₆, with 2 drops D₂O, 300 MHz): δ 7.31-7.16 (m, 4H), 2.86 (s, 2H), 1.20 (s, 6H).

### Example 4

### Preparation of 4(S)-Amino-4-[2-(4-fluoro-phenyl)-1,1-dimethyl-ethylcarbamoyl]-butyric acid tert-butyl ester

### Compound 4

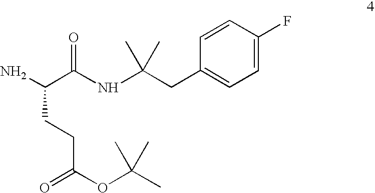

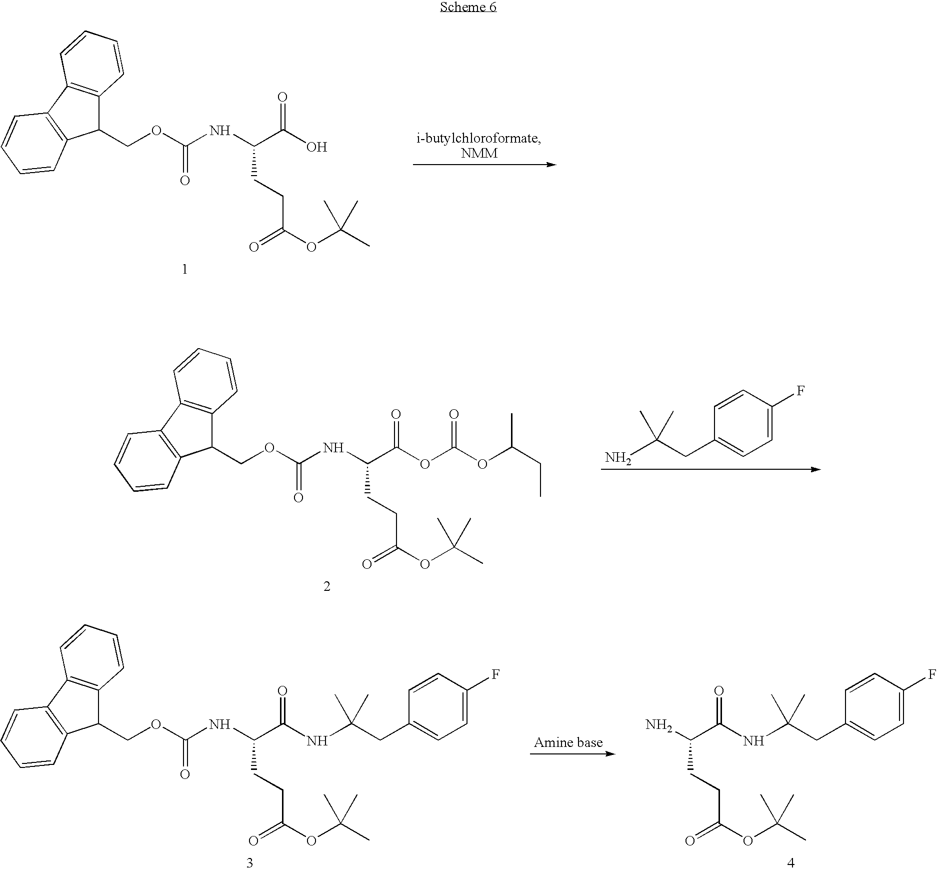

Compound 1 [2(S)-(9H-Fluoren-9-ylmethoxycarbonylamino)-pentanedioic acid 5-tert-butyl ester] (161 g) was suspended in toluene (1 L). Iso-Butyl chloroformate (59.5 g), N-methylmorpholine (91.7 g) and 2-(4-Fluoro-phenyl)-1,1-dimethyl-ethylamine (88.7 g as hydrochloride salt) were added sequentially at 5° to 15° C. After the reaction was completed in about 1 h, toluene solution was washed with water, treated with diethylamine (66.2 g) and stirred at ambient temperature until deprotection was complete (2 to 12 h). The product was extracted with 2N hydrochloric acid and by-products were removed by extraction with heptane. The resulting aqueous solution was treated with potassium carbonate and extracted with t-butyl methyl ether (TBME) to afford Compound 4 as a solution in TBME.

Alternative Synthesis of Compound 4: Compound 1 [2(S)-(9H-Fluoren-9-ylmethoxycarbonylamino)-pentanedioic acid 5-tert-butyl ester] (1 g, 2.3 mmol) was combined with THF (5 mL) and 1 drop of DMF and cooled to 0° C. Oxalyl chloride (0.328 g, 2.5 mmol) was added and the solution was stirred for about 30 min. before it was concentrated to form foam. The resulting foam was dissolved in THF and 2-(4-Fluoro-phenyl)-1,1-dimethyl-ethylamine (0.864 g, 4.6 mmol) was added. After the reaction was completed as determined by HPLC, Compound 4 was isolated following regular aqueous work-up.

### Example 5

### Preparation of 4(S)-[(Biphenyl-4-carbonyl)-amino]-4-[2-(4-fluoro-phenyl)-1,1-dimethyl-ethylcarbamoyl]-butyric acid tert-butyl ester

### Compound 5

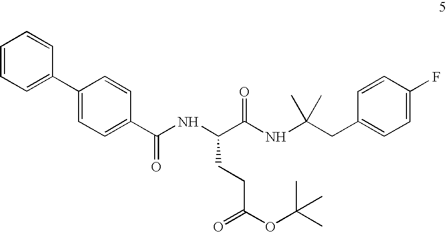

To the TBME solution of Compound 4 (561 g, strength 20%) were added triethylamine (64.6 g) and biphenyl carbonyl chloride (58.9 g, dissolved in THF) at 15° to 35° C. After the reaction was completed (1 to 18 h), the reaction mixture was washed with diluted HCl solution, sodium bicarbonate solution and water, concentrated, and Compound 5 was precipitated from the IPA/water mixture as white crystals (131 g, 77% yield). NMR data: 1.35—s, 6H, CH₃; 1.40—s, 9H, CH₃; 2.10—m, 2H, CH₂; 2.20-2.30—m, 2H, CH₂, 2.90-3.10—m, 2H, CH₂; 4.50—m, 1H, CH; 6.80-7.80—m, 13H, Ph; 7.90—d, 1H, NH.

### Example 6

### Preparation of 4(S)-[(Biphenyl-4-carbonyl)-amino]-4-[2-(4-fluoro-phenyl)-1,1-dimethyl-ethylcarbamoyl]-butyric acid

### Compound 6

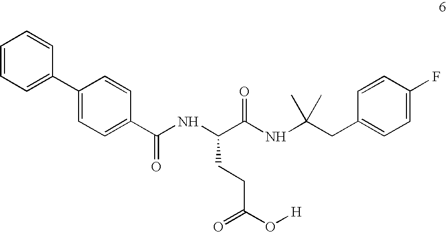

To a suspension of Compound 5 (100 g) in toluene (325 ml) were added trifluoroacetic acid (TFA, 313 g) at 5° to 20° C. The resulting solution was stirred at ambient temperature until the reaction was completed (4 to 6 h). TFA was removed by vacuum distillation, the solution diluted with ethyl acetate, washed with aqueous potassium acetate, and crystallization was affected by adding heptane to afford Compound 6 as white solid (82.7 g, yield 92%; Purity—99.8% (HPLC area %); Strength—98.0%; ee—99.0%). NMR data: 1.37, 1.45—s, 6H, CH₃; 2.10—m, 2H, CH₂; 2.35-2.60—m, 2H, CH₂; 2.80-3.10—d, 2H, CH₂; 4.80—q, 1H, CH; 6.80-7.80—m, 13H, Ph; 7.90—s, 2H, NH.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. A method for preparing a compound of formula (XIII), 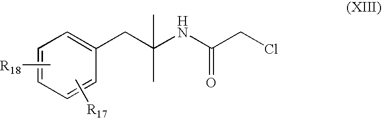
comprising:
(a) treating a compound of formula (VIII), 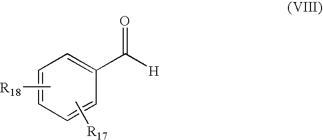
with an organometallic compound selected from the group consisting of isopropyl magnesium halide, isopropyl lithium, diisopropyl zinc and isopropyl zinc halide,
to provide a compound of formula (X); and 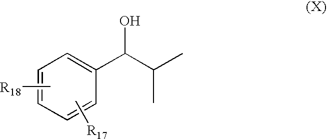
(b) treating the compound of formula (X) with at least one acid and chloroacetonitrile under conditions to effect a Ritter reaction,
wherein:
R₁₇ and R₁₈ are each independently hydrogen, halogen, —CN, —OCF₃, —CF₃, —NO₂, (C₁-C₆)alkyl, (C₂-C₆)alkenyl, (C₂-C₆)alkynyl, aryl, heteroaryl, cycloalkyl, —(CH₂)ₙR₁₁, or —O—(C₁-C₆)alkyl;
R₁₁ is aryl, heteroaryl, or cycloalkyl; and
n is 0, 1, 2, 3, or 4.

2. The method of claim 1, wherein said organometallic compound is isopropyl magnesium chloride.

3. The method of claim 1, wherein said at least one acid comprises sulfuric acid.

4. The method of claim 1, further comprising:
(a) treating the compound of formula (XIII) with a base and/or thiourea to give a compound of formula (XIV); and 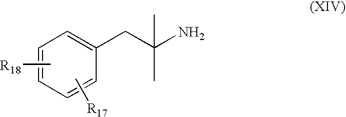
(b) optionally treating the compound of formula (XIV) with a pharmaceutically acceptable acid to provide a corresponding pharmaceutically acceptable salt of the compound of formula (XIV),
wherein R₁₇ and R₁₈ are defined as in claim 1.

5. The method of claim 4, wherein said at least one acid comprises sulfuric acid.

6. The method of claim 4, wherein said pharmaceutically acceptable acid is hydrochloric acid.

7. The method of claim 4, further comprising:
(a) treating the compound of formula (XIV) or its pharmaceutically acceptable salt with a compound selected from the group consisting of:
(i) a compound of formula (IV), 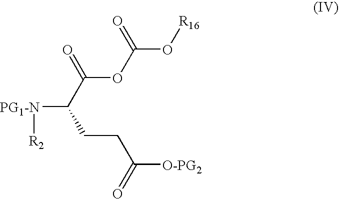
(ii) a compound of formula (IVb), and 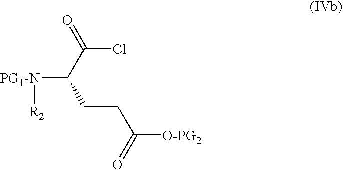
(iii) a compound of formula (II), 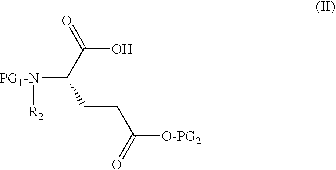
to give a compound of formula (V); 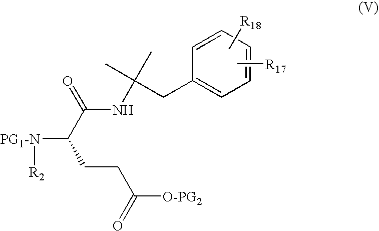
(b) removing the amine protecting group of the compound of formula (V) to give a compound of formula (VI); 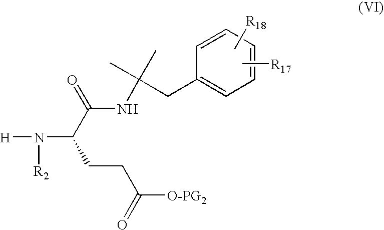
(c) treating the compound of formula (VI) with an acid chloride having the formula R₁C(═O)Cl in the presence of a base to give a compound of formula (VII); and 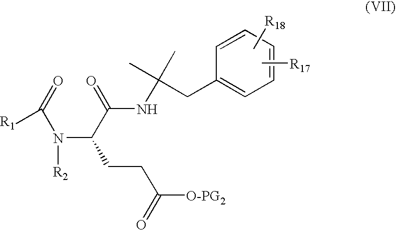
(d) removing the carboxylic acid protecting group of the compound of formula (VII) to give a compound of formula (I), 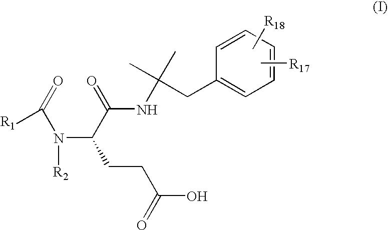
wherein:
R₁ is phenyl, heteroaryl, biphenyl, bicyclic aryl, tricyclic aryl, bicyclic heteroaryl, or tricyclic heteroaryl, each optionally substituted with one or more of R₅ or R₆, and when R₁ is substituted with more than one of R₅ or R₆, the substituents can be identical or different;
R₂ is hydrogen, (C₁-C₆) alkyl, (C₂-C₆) alkenyl, (C₂-C₆) alkynyl, —(CH₂)ₙR₁₁, —OH, or —O—(C₁-C₆) alkyl;
R₅ is aryl, heteroaryl, —(CH₂)ₙ-aryl, —(CH₂)ₙ-heteroaryl, —O-aryl, —O-heteroaryl, S-aryl, —S-heteroaryl, —NH-aryl, —NH-heteroaryl, —C(═O)—(C₁-C₆) alkyl, —C(═O)-aryl, —C(═O)-heteroaryl, —SO₂—(C₁-C₆) alkyl, —SO₂-aryl, —SO₂-heteroaryl, —SO₂NH-aryl, —SO₂NH-heteroaryl, —NHSO₂—(C₁-C₆) alkyl, —NHSO₂-aryl, —NHSO₂-heteroaryl, —NHC(═O)-aryl, —NHC(═O)-heteroaryl, —C(═O)NH-aryl, —C(═O)NH-heteroaryl, (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, or —C(═O)NH—(C₁-C₆) cycloalkyl; each alkyl, aryl, cycloalkyl, or heteroaryl optionally substituted with one or more of R₆, and when R₅ is substituted with more than one R₆, the substituents can be identical or different;
R₆ is hydrogen, halogen, —CN, —OCF₃, —CF₃, —NO₂, —OH, —SH, —NR₇R₈, —C(═O)NR₇R₈, —NR₈C(═O)R₇, —NR₈CO₂R₇, —CO₂R₇, —C(═O)R₇, —SO₂—(C₁-C₆) alkyl, —SO₂-aryl, —SO₂-heteroaryl, —SO₂R₇, —NR₇SO₂R₈, —SO₂NR₇R₈; (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, —C(═O)NH—(C₁-C₆) cycloalkyl, heterocycloalkyl, —(C₁-C₆) alkyl-OR₇, (C₂-C₆) alkynyl, (C₂-C₆) alkenyl, —O—(C₁-C₆) alkyl-cycloalkyl, —O-alkenyl, —O—(C₁-C₆) alkyl substituted with aryl, aryl, heteroaryl, —(CH₂)ₙ-aryl, —(CH₂)ₙ-heteroaryl, —O-aryl, —O-heteroaryl, —S-aryl, or —S-heteroaryl; each alkyl, aryl, cycloalkyl, heterocycloalkyl, heteroaryl, alkenyl, or alkynyl optionally substituted with one or more of R₁₃, and when R₆ is substituted with more than one R₁₃, the substituents can be identical or different;
R₇ and R₈ are each independently hydrogen, (C₁-C₆) alkyl, aryl, heteroaryl, (C₂-C₆) alkenyl, (C₂-C₆) alkynyl, cycloalkyl, —(CH₂)ₙ-aryl, or —(CH₂)ₙ-heteroaryl; or R₇ and R₈ with the nitrogen atom to which they are attached together may form a five- to seven-membered cyclic group containing up to 3 heteroatoms each independently selected from N, O, or S;
R₁₁ is aryl, heteroaryl, or cycloalkyl;
R₁₃ is halogen, —O—(C₁-C₆) alkyl, —CO₂H, —OH, —CF₃, hydrogen, (C₁-C₆) alkyl, aryl, heteroaryl, (C₂-C₆) alkenyl, (C₂-C₆) alkynyl, cycloalkyl, cycloalkyl substituted with —OH, aryl substituted with —NH₂, aryl substituted with —O—(C₁-C₆) alkyl, —(CH₂)ₙ-aryl, or —(CH₂)ₙ-heteroaryl;
R₁₆ is (C₁-C₆) alkyl;
R₁₇ and R₁₈ are as defined in claim 1;
PG₁ is an amine protecting group;
PG₂ is a carboxylic acid protecting group; and
n is 0, 1, 2, 3, or 4.

8. A method for preparing a compound of formula (XIIIa), 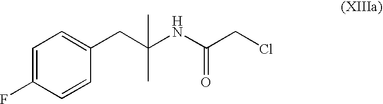
comprising:
(a) treating a compound of formula (VIIIa), 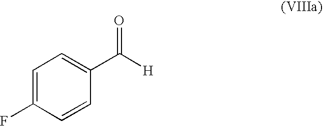
with isopropylmagnesium chloride,
to provide a compound of formula (Xa); and 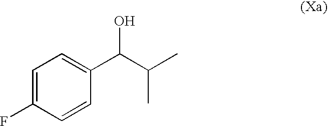
(b) treating the compound of formula (Xa) with at least one acid and chloroacetonitrile under conditions to effect a Ritter reaction.

9. The method of claim 8, wherein said at least one acid comprises sulfuric acid.

10. The method of claim 8, wherein said at least one acid comprises glacial acetic acid and sulfuric acid.

11. The method of claim 8, further comprising:
(a) treating the compound of formula (XIIIa) with a base and/or thiourea to give a compound of formula (XIVa); and 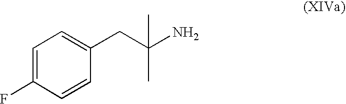
(b) optionally treating the compound of formula (XIVa) with a pharmaceutically acceptable acid to provide a corresponding pharmaceutically acceptable salt of the compound of formula (XIVa).

12. The method of claim 11, wherein in step (a), the compound of formula (XIIIa) is treated with thiourea to give a compound of formula (XIVa).

13. The method of claim 12, wherein said at least one acid comprises sulfuric acid.

14. The method of claim 12, wherein said at least one acid comprises glacial acetic acid and sulfuric acid.

15. The method of claim 12, wherein said pharmaceutically acceptable acid is hydrochloric acid.

16. The method of claim 11, further comprising converting the compound having formula (XIVa) or its pharmaceutically acceptable salt into a compound of formula (Ia) or a pharmaceutically acceptable salt thereof,

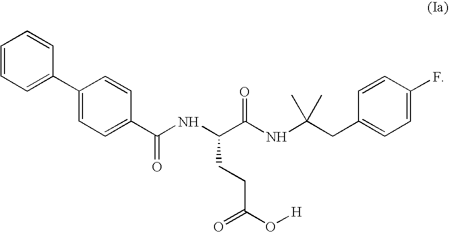

17. The method of claim 11, further comprising:
(a) treating the compound of formula (XIVa) or its pharmaceutically acceptable salt with a compound selected from the group consisting of:
(i) a compound of formula (IVa), 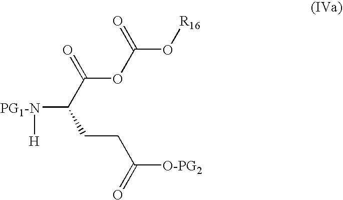
(ii) a compound of formula (IVc), and 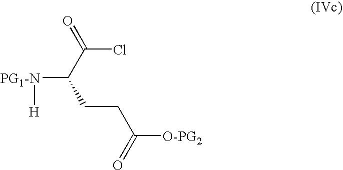
(iii) a compound of formula (IIa), 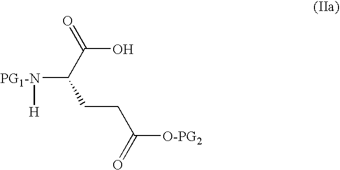
to give a compound of formula (Va); 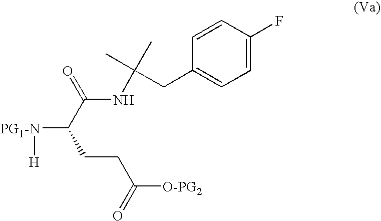
(b) removing the amine protecting group of the compound of formula (Va) to give a compound of formula (VIa); 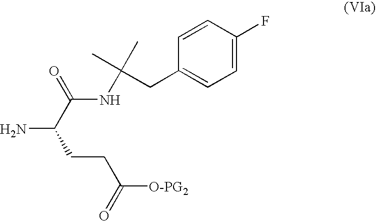
(c) treating the compound of formula (VIa) with an acid chloride having the formula of 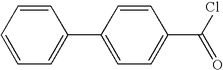
in the presence of a base to give a compound of formula (VIIa); and 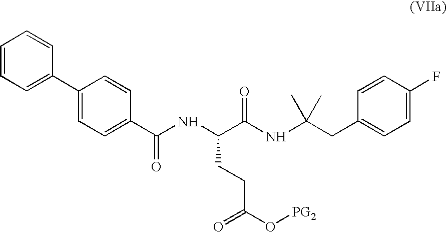
(d) removing the carboxylic acid protecting group of the compound of formula (VIIa) to give a compound of formula (Ia), 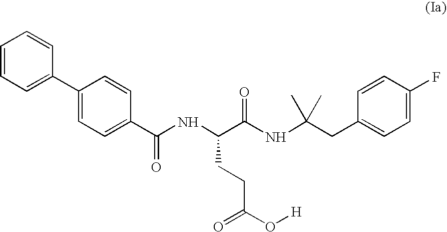
wherein:
PG₁ is an amine protecting group;
PG₂ is a carboxylic acid protecting group; and
R₁₆ is (C₁-C₆) alkyl.

18. A compound of formula (XIIIa)

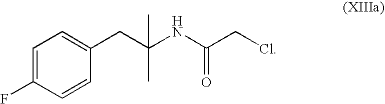

19. A compound of formula (XIII), 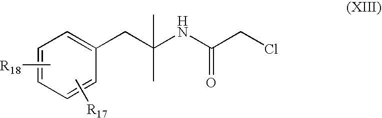
prepared by a method according to claim 1, and wherein R₁₇ and R₁₈ are defined as in claim 1.

20. A compound of formula (XIV), or a pharmaceutically acceptable salt thereof, 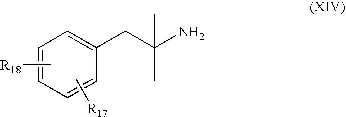
prepared by a method according to claim 4, and wherein R₁₇ and R₁₈ are defined as in claim 1.

21. A compound of formula (I), 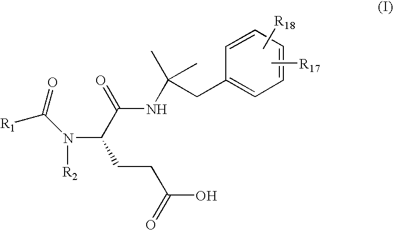
prepared by a method according to claim 7, and wherein R₁ and R₂ are defined as in claim 7; and R₁₇ and R₁₈ are defined as in claim 1.

22. A compound of formula (XIIIa), 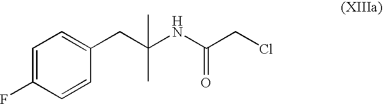
prepared by a method according to claim 8.

23. A compound of formula (XIVa), or a pharmaceutically acceptable salt thereof, 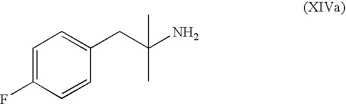
prepared by a method of according to claim 11.

24. The compound of claim 23, wherein said pharmaceutically acceptable salt is hydrochloric acid salt.

25. A compound of formula (Ia), 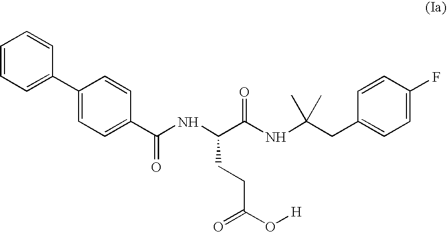
prepared by a method according to claim 16.

26. A method of preparing a compound of formula (I) or a pharmaceutically acceptable salt thereof, 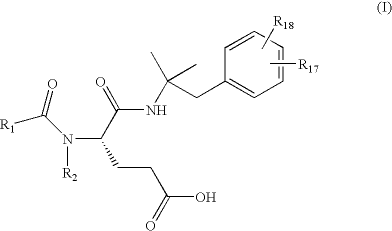
comprising:
(a) treating a compound of formula (VIII), 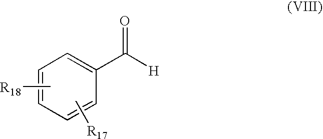
with an organometallic compound selected from the group consisting of isopropyl magnesium halide, isopropyl lithium, diisopropyl zinc and isopropyl zinc halide, preferably isopropyl magnesium chloride,
to provide a compound of formula (X); 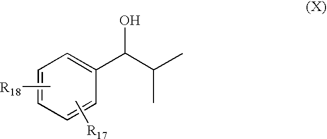
(b) treating the compound of formula (X) with at least one acid and chloroacetonitrile under conditions to effect a Ritter reaction to give a compound of formula (XIII), 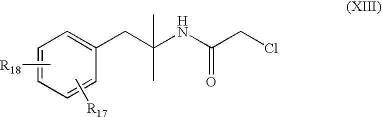
(c) treating the compound of formula (XIII) with a base and/or thiourea to give a compound of formula (XIV); 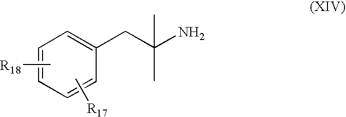
(d) optionally treating the compound of formula (XIV) with a pharmaceutically acceptable acid to provide a corresponding pharmaceutically acceptable salt of the compound of formula (XIV);
(e) converting the compound having formula (XIV) or its pharmaceutically acceptable salt into the compound of formula (I) or a pharmaceutically acceptable salt thereof,
wherein:
R₁ is phenyl, heteroaryl, biphenyl, bicyclic aryl, tricyclic aryl, bicyclic heteroaryl, or tricyclic heteroaryl, each optionally substituted with one or more of R₅ or R₆, and when R₁ is substituted with more than one of R₅ or R₆, the substituents can be identical or different;
R₂ is hydrogen, (C₁-C₆) alkyl, (C₂-C₆) alkenyl, (C₂-C₆) alkynyl, —(CH₂)ₙR₁₁, —OH, or —O—(C₁-C₆) alkyl;
R₅ is aryl, heteroaryl, —(CH₂)ₙ-aryl, —(CH₂)ₙ-heteroaryl, —O-aryl, —O-heteroaryl, —S-aryl, —S-heteroaryl, —NH-aryl, —NH-heteroaryl, —C(═O)—(C₁-C₆) alkyl, —C(═O)-aryl, —C(═O)-heteroaryl, —SO₂—(C₁-C₆) alkyl, —SO₂-aryl, —SO₂-heteroaryl, —SO₂NH-aryl, —SO₂NH-heteroaryl, —NHSO₂—(C₁-C₆) alkyl, —NHSO₂-aryl, —NHSO₂-heteroaryl, —NHC(═O)-aryl, —NHC(═O)-heteroaryl, —C(═O)NH-aryl, —C(═O)NH-heteroaryl, (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, or —C(═O)NH—(C₁-C₆) cycloalkyl; each alkyl, aryl, cycloalkyl, or heteroaryl optionally substituted with one or more of R₆, and when R₅ is substituted with more than one R₆, the substituents can be identical or different;
R₆ is hydrogen, halogen, —CN, —OCF₃, —CF₃, —NO₂, —OH, —SH, —NR₇R₈, —C(═O)NR₇R₈, —NR₈C(═O)R₇, —NR₈CO₂R₇, —CO₂R₇, —C(═O)R₇, —SO₂—(C₁-C₆) alkyl, —SO₂-aryl, —SO₂-heteroaryl, —SO₂R₇, —NR₇SO₂R₈, —SO₂NR₇R₈; (C₁-C₆) alkyl, —O—(C₁-C₆) alkyl, —S—(C₁-C₆) alkyl, —NH—(C₁-C₆) alkyl, —NHC(═O)—(C₁-C₆) alkyl, —C(═O)NH—(C₁-C₆) alkyl, —O—(C₁-C₆) cycloalkyl, —S—(C₁-C₆) cycloalkyl, —NH—(C₁-C₆) cycloalkyl, —NHC(═O)—(C₁-C₆) cycloalkyl, —C(═O)NH—(C₁-C₆) cycloalkyl, heterocycloalkyl, —(C₁-C₆) alkyl-OR₇, (C₂-C₆) alkynyl, (C₂-C₆) alkenyl, —O—(C₁-C₆) alkyl-cycloalkyl, —O-alkenyl, —O—(C₁-C₆) alkyl substituted with aryl, aryl, heteroaryl, —(CH₂)ₙ-aryl, —(CH₂)ₙ-heteroaryl, —O-aryl, —O-heteroaryl, —S-aryl, or —S-heteroaryl; each alkyl, aryl, cycloalkyl, heterocycloalkyl, heteroaryl, alkenyl, or alkynyl optionally substituted with one or more of R₁₃, and when R₆ is substituted with more than one R₁₃, the substituents can be identical or different;
R₇ and R₈ are each independently hydrogen, (C₁-C₆) alkyl, aryl, heteroaryl, (C₂-C₆) alkenyl, (C₂-C₆) alkynyl, cycloalkyl, —(CH₂)ₙ-aryl, or —(CH₂)ₙ-heteroaryl; or R₇ and R₈ together may form a five- to seven-membered cyclic group containing up to 3 heteroatoms selected from N, O, or S;
R₁₃ is halogen, —O—(C₁-C₆) alkyl, —CO₂H, —OH, —CF₃, hydrogen, (C₁-C₆) alkyl, aryl, heteroaryl, (C₂-C₆) alkenyl, (C₂-C₆) alkynyl, cycloalkyl, cycloalkyl substituted with —OH, aryl substituted with —NH₂, aryl substituted with —O—(C₁-C₆) alkyl, —(CH₂)ₙ-aryl, or —(CH₂)ₙ-heteroaryl;
R₁₇ and R₁₈ are each independently hydrogen, halogen, —CN, —OCF₃, —CF₃, —NO₂, (C₁-C₆)alkyl, (C₂-C₆)alkenyl, (C₂-C₆)alkynyl, aryl, heteroaryl, cycloalkyl, —(CH₂)ₙR₁₁, or —O—(C₁-C₆)alkyl;
R₁₁ is aryl, heteroaryl, or cycloalkyl; and
n is 0, 1, 2, 3, or 4.
